(19) United States
(12) Patent Application Publication
(10) Pub. No.: US 2024/0368548 A1
(43) Pub. Date: Nov. 7, 2024

(54) Methods for Producing Regulatory T Cell (Treg) Populations, Treg Compositions and Methods for Treatment

(71) Applicants: The Methodist Hospital, Houston, TX (US); Coya Therapeutics, Inc., Houston, TX (US)

(72) Inventors: Stanley Hersh Appel, Houston, TX (US); Jason Robert Thonhoff, Houston, TX (US); David Robert Beers, Houston, TX (US); Aaron Drew Thome, Richmond, TX (US); Weihua Zhao, Pearland, TX (US); Howard Berman, Houston, TX (US)

(21) Appl. No.: 18/568,142

(22) PCT Filed: Jun. 7, 2022
(86) PCT No.: PCT/US2022/032474

§ 371 Date: Dec. 7, 2023

Related U.S. Application Data
(60) Provisional application No. 63/208,393, filed on Jun. 8, 2021.
(60) Provisional application No. 63/314,147, filed on Feb. 25, 2022.

(51) Int. Cl. C12N 5/0783; A61K 35/17

(52) U.S. Cl. CPC C12N 5/0637; A61K 35/17; C12N 2501/2302; C12N 2501/51; C12N 2501/515; C12N 2501/999

(57) ABSTRACT

Disclosed are improved, bioreactor-based methods for manufacturing large-scale populations of robust, highly pure, and functional T regulatory cells (Tregs). Also disclosed are expanded Treg populations, cryopreserved Treg populations and methods and uses of these cells in compositions formulated for treating one or more mammalian diseases, including, for example, treatment, prophylaxis, and/or amelioration of one or more symptoms of a human neurodegenerative disorder. In particular, the compositions and methods provided herein find clinical use in the treatment and amelioration of one or more symptoms of amyotrophic lateral sclerosis (ALS), Alzheimer's disease, and other neurological diseases and disorders.

### 1. CROSS REFERENCE TO RELATED APPLICATIONS

This application claims the benefit of priority from both U.S. Provisional Application No. 63/208,393, filed Jun. 8, 2021, and U.S. Provisional Application No. 63/314,147, filed Feb. 25, 2022. All of the foregoing related applications, in their entirety, are incorporated herein by reference.

### 2. FIELD

The present disclosure relates to the fields of medicine, molecular biology, and specifically to the manufacture of medicaments suitable for use in the treatment of mammalian neurodegenerative diseases. In particular, the disclosure provides improved methods for manufacturing robust, highly pure, and functional T regulatory cells useful in the treatment of diseases such as amyotrophic lateral sclerosis (ALS), Alzheimer's disease, and other neurological, as well as inflammatory and autoimmune diseases or dysfunctions.

### 3. BACKGROUND

The clinical manufacturing of expanded autologous CD4⁺CD25^{high}FOXP3⁺ T regulatory cells (Tregs) presents several logistical and cost-related challenges that impede the availability of these therapies to patients who may benefit from their use. Current Treg manufacturing conditions require activation and expansion protocols that are complicated and labor-intensive. Further challenges include the time required to expand the Tregs to the necessary doses and the formulation of a final cryopreserved product after expansion that maintains cellular viability, integrity and function. Despite these challenges, autologous Treg therapies are currently being tested in Phase I clinical studies for Graft-Versus-Host Disease (GvHD) and several autoimmune diseases including type 1 diabetes and Crohn's disease. The therapeutic value of increasing Treg activity is supported by the fact that the efficacy of many immunosuppressive drugs is contingent upon their abilities to stimulate Tregs. Treg therapies may be more advantageous than immunosuppressant drugs as they may limit off target effects, thus improving efficacy and minimizing adverse effects. Therefore, the development of a manufacturing process for the robust production of highly pure and functional Tregs is crucial for future applications of Treg therapies.

### 4. SUMMARY

Treg adoptive cell therapies hold great promise for treating patients with a wide variety of disorders. A challenge to fulfilling the potential of such therapies, however, is to be able to efficiently and quickly produce large numbers of Tregs that exhibit high purity, viability and suppressive potency that can be stored and ready for administration to patients. The methods presented herein address this challenge and provide the ability to produce potent ex vivo-expanded Treg cell populations that may be utilized as off-the-shelf therapeutics.

Also presented herein are ex vivo-expanded Treg cell populations, pharmaceutical compositions comprising such Treg cell populations, cryopreserved ex vivo-expanded therapeutic Treg populations, and pharmaceutical compositions comprising such cryopreserved Tregs following their thawing and without further expansion.

Also presented herein are methods of treatment that utilize the Treg cell populations produced and described herein, including, for example, treatment of neurodegenerative disorders such as amyotrophic lateral sclerosis (ALS), Alzheimer's disease, Parkinson's disease and frontotemporal dementia.

In one aspect, provide herein is a method of producing a cryopreserved therapeutic population of regulatory T cells (Tregs), said method comprising the steps of. (a) enriching Tregs from a cell sample suspected of containing Tregs, to produce a baseline Treg cell population; (b) expanding the baseline Treg cell population to produce an expanded Treg cell population, wherein the baseline Treg cell population is not cryopreserved prior to the initiation of step (b), and wherein step (b) takes place in a bioreactor; and (c) cryopreserving the expanded Treg cell population to produce a cryopreserved therapeutic population of Tregs. In this context, the term “baseline,” or “baseline Treg cell population” denotes a population of Tregs that has been enriched from a patient sample but has not yet been expanded. In certain embodiments, step (a) comprises depleting CD8+/CD19+ cells then enriching for CD25+ cells. In certain embodiments, step (b) is carried out within about 30 minutes after step (a).

In certain embodiments, step (b) comprises culturing the Tregs in a culture medium that comprises beads coated with anti-CD3 antibodies and anti-CD28 antibodies. In specific embodiments, step (b) comprises adding the beads to the culture medium within about 24 hours of the initiation of the culturing. In specific embodiments, step (b) comprises adding the beads to the culture medium within about 30 minutes after the completion of step (a). In specific embodiments, step (b) comprises adding beads coated with anti-CD3 antibodies and anti-CD28 antibodies to the culture medium about 11 days after beads coated with anti-CD3 antibodies and anti-CD28 antibodies are first added to the culture medium. In specific embodiments, step (b) comprises adding beads coated with anti-CD3 antibodies and anti-CD28 antibodies to the culture medium about 11 days after beads coated with anti-CD3 antibodies and anti-CD28 antibodies are first added to the culture medium, if the cell number by then has not reached a target cell number. The cell number means the number of all cells in culture, including the enriched Treg cells, which represent a majority of the cells in culture and in specific embodiments represent more than 70%, more than 80%, more than 90%, more than 95%, more than 99%, or 100% of the cells in culture. In a specific embodiment, the target cell number is 2.5×10⁹ cells.

In certain embodiments, step (b) comprises culturing the Tregs in a culture medium that comprises Interleukin-2 (IL-2). In specific embodiments, step (b) comprises adding IL-2 to the culture medium within about 24 hours of the initiation of the culturing. In specific embodiments, step (b) comprises adding IL-2 to the culture medium within about 30 minutes after the completion of step (a). In specific embodiments, step (b) comprises replenishing the culture medium with IL-2 about every 3-4 days after IL-2 is first added to the culture medium. In specific embodiments, step (b) comprises adjusting IL-2 concentration depending on cell number. The cell number means the number of all cells in culture, including the enriched Treg cells, which represent a majority of the cells in culture and in specific embodiments represent more than 70%, more than 80%, more than 90%, more than 95%, more than 99%, or 100% of the cells in culture. In a specific embodiment, step (b) comprises culturing the Tregs in a culture medium containing about 200 IU/mL IL-2 until the cell number reaches 600×10⁶, and then culturing the Tregs in a culture medium containing about 250 IU/mL IL-2.

In certain embodiments, step (b) comprises culturing the Tregs in a culture medium that comprises rapamycin. In specific embodiments, step (b) comprises adding rapamycin to the culture medium within about 24 hours of the initiation of the culturing. In specific embodiments, step (b) comprises adding rapamycin to the culture medium within about 30 minutes after the completion of step (a).

In certain embodiments, step (b) comprises adjusting flow rate of an extracapillary (EC) medium of the bioreactor depending on cell number. The cell number means the number of all cells in culture, including the enriched Treg cells, which represent a majority of the cells in culture and in specific embodiments represent more than 70%, more than 80%, more than 90%, more than 95%, more than 99%, or 100% of the cells in culture. In specific embodiments, the extracapillary medium comprises rapamycin. In specific embodiments, step (b) comprises maintaining the flow rate of the EC medium at 0 until the cell number reaches 500×10⁶, then increasing the flow rate of the EC medium to about 0.2 mL/min and maintaining the flow rate of the EC medium at about 0.2 mL/min until the cell number reaches 750×10⁶, then increasing the flow rate of the EC medium to about 0.4 mL/min and maintaining the flow rate of the EC medium at about 0.4 mL/min until the cell number reaches about 1,000×10⁶, then increasing the flow rate of the EC medium to about 0.6 mL/min and maintaining the flow rate of the EC medium at about 0.6 mL/min until the cell number reaches about 1,500×10⁶, and then increasing the flow rate of the EC medium to about 0.8 mL/min and maintaining the flow rate of the EC medium at about 0.8 mL/min.

In certain embodiments, the cell sample is a leukapheresis cell sample.

In certain embodiments, step (b) is automated. In certain embodiments, step (b) takes place in a closed system. In certain embodiments, step (a) is automated. In certain embodiments, step (a) takes place in a closed system. In some embodiments, step (a) and step (b) take place in different systems. In specifics embodiments, the baseline Treg cell population produced by step (a) are transferred to the bioreactor in step (b) in a closed step. In specific embodiments, step (a) takes place in a closed system, step (b) takes place in a closed system, and the baseline Treg cell population produced by step (a) are transferred to the bioreactor in step (b) in a closed step. In other embodiments, step (a) and step (b) take place in the same system. In specific embodiments, the same system is a closed system.

In certain embodiments, the method further comprises thawing the cryopreserved therapeutic population of Tregs and, without further expansion, placing the population into a composition comprising a pharmaceutically acceptable carrier, to produce a pharmaceutical composition. In specific embodiments, the method further comprises administering the pharmaceutical composition to a subject. In some embodiments, the Tregs in the pharmaceutical composition are autologous to the subject. In other embodiments, the Tregs in the pharmaceutical composition are allogeneic to the subject. In various embodiments, the subject is a human subject.

In another aspect, provided herein is a cryopreserved therapeutic population of Tregs produced by a method described herein.

In another aspect, provided herein is a pharmaceutical composition comprising a thawed and unexpanded form of a cryopreserved therapeutic population of Tregs described herein, and a pharmaceutically acceptable carrier.

In another aspect, provided herein is a pharmaceutical composition produced by a method described herein.

In another aspect, provided herein is a method of treating a disorder associated with Treg dysfunction in a subject in need thereof, the method comprising administering to the subject a pharmaceutical composition described herein.

In another aspect, provided herein is a method of treating a disorder associated with Treg deficiency in a subject in need thereof, the method comprising administering to the subject a pharmaceutical composition described herein.

In another aspect, provided herein is a method of treating a disorder associated with overactivation of the immune system in a subject in need thereof, the method comprising administering to the subject a pharmaceutical composition described herein.

In another aspect, provided herein is a method of treating an inflammatory condition driven by a T cell response in a subject in need thereof, the method comprising administering to the subject a pharmaceutical composition described herein.

In another aspect, provided herein is a method of treating an inflammatory condition driven by a myeloid cell response in a subject in need thereof, the method comprising administering to the subject a pharmaceutical composition described herein. In specific embodiments, the myeloid cell is a monocyte, macrophage or microglia.

In another aspect, provided herein is a method of treating a neurodegenerative disorder in a subject in need thereof, the method comprising administering to the subject a pharmaceutical composition described herein. In specific embodiments, the neurodegenerative disease is Amyotrophic Lateral Sclerosis (ALS), Alzheimer's disease, Parkinson's disease, frontotemporal dementia or Huntington's disease.

In another aspect, provided herein is a method of treating an autoimmune disorder in a subject in need thereof, the method comprising administering to the subject a pharmaceutical composition described herein. In specific embodiments, the autoimmune disorder is polymyositis, ulcerative colitis, inflammatory bowel disease, Crohn's disease, celiac disease, systemic sclerosis (scleroderma), multiple sclerosis (MS), rheumatoid arthritis (RA), Type I diabetes, psoriasis, dermatomyosititis, lupus, e.g., systemic lupus erythematosus, or cutaneous lupus, myasthenia gravis, autoimmune nephropathy, autoimmune hemolytic anemia, autoimmune cytopenia, autoimmune encephalitis, autoimmune hepatitis, autoimmune uveitis, alopecia, thyroiditis or pemphigus.

In another aspect, provided herein is a method of treating graft-versus-host disease in a subject in need thereof, the method comprising administering to the subject a pharmaceutical composition described herein. In specific embodiments, the subject has received a bone marrow transplant, kidney transplant or liver transplant.

In another aspect, provided herein is a method of improving islet graft survival in a subject in need thereof, comprising administering to the subject a pharmaceutical composition described herein in combination with the islet transplantation.

In another aspect, provided herein is a method of treating cardio-inflammation in a subject in need thereof, the method comprising administering to the subject a pharmaceutical composition described herein. In specific embodiments, the cardio-inflammation is associated with atherosclerosis, myocardial infarction, ischemic cardiomyopathy or heart failure.

In another aspect, provided herein is a method of treating neuroinflammation in a subject in need thereof, the method comprising administering to the subject a pharmaceutical composition described herein. In specific embodiments, the neuroinflammation is associated with stroke, acute disseminated encephalomyelitis, acute optic neuritis, acute inflammatory demyelinating polyradiculoneuropathy, chronic inflammatory demyelinating polyradiculoneuropathy, Guillain-Barre syndrome, transverse myelitis, neuromyelitis optica, epilepsy, traumatic brain injury, spinal cord injury, encephalitis, central nervous system vasculitis, neurosarcoidosis, autoimmune or post-infectious encephalitis or chronic meningitis.

In another aspect, provided herein is a method of treating a Tregopathy in a subject in need thereof, comprising administering to the subject a pharmaceutical composition described herein. In specific embodiments, the Tregopathy is caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

In some embodiments, the expanded Treg cell population and the cryopreserved therapeutic population of Tregs, following thawing and without additional expansion, exhibit an ability to suppress inflammatory cells, as measured by pro-inflammatory cytokine production by the inflammatory cells, wherein the inflammatory cells are macrophages or monocytes from human donors or generated from induced pluripotent stem cells. In some embodiments, the ability of the cryopreserved therapeutic population of Tregs, following thawing and without additional expansion, to suppress inflammatory cells is at least 70% that of the expanded Treg cell population.

In some embodiments, the ability to suppress inflammatory cells is measured by IL-6, TNFα, IL1β, IL8, and/or Interferon-γ production by the inflammatory cells. In some embodiments, the ability to suppress inflammatory cells is measured by IL-6 production by the inflammatory cells. In some embodiments, the cryopreserved therapeutic population of Tregs, following thawing and without additional expansion, exhibits a suppressive function, wherein the suppressive function is greater than that of the baseline Treg cell population, as determined by suppression of proliferation of responder T cells. In some embodiments, the suppressive function of the cryopreserved therapeutic population of Tregs, following thawing and without additional expansion, is at least 25%, at least 50%, at least 75%, at least 100% at least 150%, or at least 300% greater than the suppressive function of the baseline Treg cell population as determined by suppression of proliferation of responder T cells. In some embodiments, the cryopreserved therapeutic population of Tregs, following thawing and without additional expansion, exhibits a suppressive function, wherein the suppressive function is at least 50%, at least 60%, at least 70%, at least 75%, at least 80%, at least 85%, at least 90% or at least 95%, as determined by suppression of proliferation of responder T cells. In some embodiments, the suppressive function of the cryopreserved therapeutic population of Tregs, following thawing and without additional expansion, is at least 50%, at least 60%, at least 70%, at least 75%, at least 80%, at least 85%, at least 90% or at least 95% of the suppressive function of the expanded Treg cell population before cryopreservation. In some embodiments, the proliferation of responder T cells is determined by flow cytometry or thymidine incorporation.

In some embodiments, the viability of the cryopreserved therapeutic population of Tregs, following thawing and without additional expansion, is at least 70%, at least 75%, at least 80%, at least 85%, at least 90% or at least 95%, as determined by trypan blue staining. In some embodiments, the viability of the cryopreserved therapeutic population of Tregs, following thawing and without additional expansion, is at least 70%, at least 75%, at least 80%, at least 85%, at least 90% or at least 95% of the viability of the expanded Treg cell population prior to the expanded Treg cell population being cryopreserved in step (c), as determined by trypan blue staining.

In some embodiments, the cryopreserved therapeutic population of Tregs comprises FoxP3+ Tregs wherein the proportion of FoxP3+ Tregs is increased relative to the proportion of FoxP3+ Tregs in the Tregs in the baseline Treg cell population. In some embodiments, the cryopreserved therapeutic population of Tregs comprises at least 40%, at least 50%, at least 60%, at least 70%, or at least 80% or at least 90% FoxP3+ Tregs, as determined by flow cytometry. In some embodiments, the cryopreserved therapeutic population of Tregs comprise FoxP3-expressing Tregs wherein the expression of FoxP3 is increased in the Tregs relative to expression of FoxP3 in the Tregs in the baseline Treg cell population.

In some embodiments, the cryopreserved therapeutic population of Tregs comprises at least 70%, at least 75%, at least 80%, at least 85%, at least 90% or at least 95% CD4⁺CD25⁺ cells, as determined by flow cytometry. In some embodiments, the cryopreserved therapeutic population of Tregs comprises fewer than 20% CD8+ cells, as determined by flow cytometry. In some embodiments, the cryopreserved therapeutic population of Tregs comprises at least 70%, at least 80%, or at least 90% CD4⁺CD25^{high} CD127^{low} Tregs, as determined by flow cytometry.

In some embodiments, the cell sample is a leukapheresis cell sample. In some embodiments, the method further comprises obtaining the cell sample from a donor by leukapheresis. In some embodiments, the cell sample is not stored overnight or frozen before carrying out the enriching step (a). In some embodiments, the cell sample is obtained within 30 minutes before initiation of enriching step (a). In some embodiments, step (a) comprises depleting CD8+/CD19+ cells then enriching for CD25+ cells. In some embodiments, step (b) is carried out within 30 minutes after step (a).

In some embodiments, step (b) comprises culturing the Tregs in a culture medium that comprises beads coated with anti-CD3 antibodies and anti-CD28 antibodies. In some embodiments, the beads are first added to the culture medium within about 24 hours of the initiation of the culturing. In some embodiments, beads coated with anti-CD3 antibodies and anti-CD28 antibodies are added to the culture medium about 14 days after beads coated with anti-CD3 antibodies and anti-CD28 antibodies were first added to the culture medium.

In some embodiments, step (b) further comprises adding IL-2 to the culture medium within about 6 days of the initiation of culturing. In some embodiments, step (b) further comprises replenishing the culture medium with IL-2 about every 2-3 days after IL-2 is first added to the culture medium.

In some embodiments, step (b) further comprises adding rapamycin to the culture medium within about 24 hours of the initiation of the culturing. In some embodiments, step (b) further comprises replenishing the culture medium with rapamycin every 2-3 days after the rapamycin is first added to the culture medium.

In some embodiments, the cryopreserving step (c) is carried out at least 6 days following IL-2 addition to or replenishment of the culture medium in step (b). In some embodiments, the cryopreserving step (c) is carried out about 8-25 days after the initiation of the culturing step (b).

In some embodiments, step(c) comprises cryopreserving the Tregs in a cryoprotectant comprising DMSO. In some embodiments, the cryopreservation step (c) comprises changing the temperature of the population of Tregs in the following increments: 1° C./min to 4° C., 25° C./min to −40° C., 10° C./min to −12° C., 1° C./min to −40° C., and 10° C./min to −80° C.-−90° C. In some embodiments, the cryopreserved therapeutic population of Tregs is frozen at a Treg density of at least 50 million cells/mL. In some embodiments, the cryopreserved therapeutic population of Tregs is frozen in a total volume of 1-1.5 mL. In some embodiments, the method further comprises thawing the cryopreserved therapeutic population of Tregs after cryopreservation for about 1 week, 1 month, about 3 months, about 6 months, about 9 months, about 12 months, about 18 months or about 24 months.

In some embodiments, the cell sample is from a human donor. In some embodiments, the human donor is a healthy donor. In other embodiments, the human donor is diagnosed with or suspected of having a neurodegenerative disorder. In some embodiments, the neurodegenerative disorder is amyotrophic lateral sclerosis, Alzheimer's disease, Parkinson's disease or frontotemporal dementia.

In some embodiments, the population of Tregs is subjected to genetic engineering at any point of the method prior to cryopreserving step (c).

In some embodiments, step (b) is automated. In some embodiments, step (b) takes place in a bioreactor. In some embodiments, In some embodiments, step (b) takes place in a G-REX culture system. In some embodiments, the method is performed in a closed system.

In some embodiments, the method further comprises thawing the cryopreserved therapeutic population of Tregs and, without further expansion, placing the population into a pharmaceutical composition comprising a pharmaceutically acceptable carrier, to produce a Treg pharmaceutical composition. In some embodiments, the Treg pharmaceutical composition comprises normal saline and 5% human serum albumin.

In some embodiments, the method further comprises administering the Treg pharmaceutical composition to a human subject. In some embodiments, the Tregs in the pharmaceutical composition are autologous to the human subject. In some embodiments, the human subject has been diagnosed with or is suspected of having a neurodegenerative disorder. In some embodiments, the neurodegenerative disorder is amyotrophic lateral sclerosis (ALS), Alzheimer's disease, Parkinson's disease or frontotemporal dementia.

In another aspect, provided herein is a cryopreserved therapeutic population of Tregs produced by the method provided herein.

In another aspect, provided herein is a pharmaceutical composition comprising a cryopreserved therapeutic population of Tregs produced by a method provided herein, following thawing and without further expansion, and a pharmaceutically acceptable carrier.

In another aspect, provided herein is an ex vivo-expanded Treg cell population that exhibits an ability to suppress inflammatory cells, as measured by pro-inflammatory cytokine production by the inflammatory cells, wherein the inflammatory cells are macrophages or monocytes from human donors or generated from induced pluripotent stem cells. In some embodiments, the ability to suppress inflammatory cells is measured by IL-6, TNFα, IL1β, IL8, and/or Interferon-γ production by the inflammatory cells. In some embodiments, the ability to suppress inflammatory cells is measured by IL-6 production by the inflammatory cells. In some embodiments, the Treg cell population is autologous to a human subject with ALS. In some embodiments, the Treg cell population has been expanded from a cell sample from a human subject with ALS.

In another aspect, provided herein is a pharmaceutical composition comprising an ex vivo-expanded Treg cell population provided herein and a pharmaceutically acceptable carrier.

In another aspect, provided herein is a cryopreserved therapeutic population of ex vivo-expanded Tregs that, following thawing and without additional expansion, exhibits an ability to suppress inflammatory cells, as measured by pro-inflammatory cytokine production by the inflammatory cells, wherein the inflammatory cells are macrophages or monocytes from human donors or generated from induced pluripotent stem cells. In some embodiments, the ability of the cryopreserved therapeutic population of ex vivo-expanded Tregs to suppress inflammatory cells, following expansion and without additional expansion, is at least 70%, that of the ex vivo-expanded Tregs prior to cryopreservation. In some embodiments, the ability to suppress inflammatory cells is measured by IL-6, TNFα, IL1β, IL8, and/or Interferon-γ production by the inflammatory cells. In some embodiments, the ability to suppress inflammatory cells is measured by IL-6 production by the inflammatory cells.

In some embodiments, the cryopreserved therapeutic population of ex vivo-expanded Tregs, following thawing and without additional expansion, exhibits a suppressive function that is at least 50%, at least 60%, at least 70%, at least 75%, at least 80%, at least 85%, at least 90% or at least 95% as determined by suppression of proliferation of responder T cells by flow cytometry or thymidine incorporation. In some embodiments, the cryopreserved therapeutic population of ex vivo-expanded Tregs, following thawing and without additional expansion, exhibits a suppressive function that is at least 50%, at least 60%, at least 70%, at least 75%, at least 80%, at least 85%, at least 90% or at least 95% of the suppressive function of the ex vivo-expanded Tregs prior to cryopreservation, as determined by suppression of proliferation of responder T cells by flow cytometry or thymidine incorporation. In some embodiments, the cryopreserved therapeutic population of ex vivo-expanded Tregs, following thawing and without additional expansion, exhibits at least 70%, at least 75%, at least 80%, at least 85%, at least 90% or at least 95% viability, as determined by trypan blue staining. In some embodiments, the cryopreserved therapeutic population of ex vivo-expanded Tregs, following thawing and without additional expansion, exhibits a viability that is at least 70%, at least 75%, at least 80%, at least 85%, at least 90% or at least 95% of the viability of the Tregs before cryopreservation, as determined by trypan blue staining.

In some embodiments, the cryopreserved therapeutic population of ex vivo-expanded Tregs, following thawing and without additional expansion, comprises at least 40%, at least 50%, at least 60%, at least 70%, or at least 80% or at least 90% FoxP3+ Tregs, as determined by flow cytometry. In some embodiments, the cryopreserved therapeutic population of ex vivo-expanded Tregs, following thawing and without additional expansion, comprises at least 70%, at least 75%, at least 80%, at least 85%, at least 90% or at least 95% CD4⁺CD25+ cells, as determined by flow cytometry. In some embodiments, the cryopreserved therapeutic population of ex vivo-expanded Tregs, following thawing and without additional expansion, comprises fewer than 20% CD8+ cells, as determined by flow cytometry. In some embodiments, the cryopreserved therapeutic population of ex vivo-expanded Tregs, following thawing and without additional expansion, comprises at least 70%, at least 80%, or at least 90% CD4⁺CD25^{high}CD127^{low} Tregs, as determined by flow cytometry. In some embodiments, the ex vivo-expanded Tregs are autologous to a human subject with ALS. In some embodiments, the ex vivo-expanded Tregs have been expanded from a cell sample from a human subject with ALS.

In another aspect, provided herein is a pharmaceutical composition comprising the cryopreserved therapeutic population of Tregs provided herein, following thawing and without further expansion, and a pharmaceutically acceptable carrier.

In some embodiments, the ex vivo-expanded Treg cell population exhibits a suppressive function, wherein the suppressive function is at least 50%, at least 60%, at least 70%, at least 75%, at least 80%, at least 85%, at least 90% or at least 95% as determined by suppression of proliferation of responder T cells by flow cytometry or thymidine incorporation. In some embodiments, the ex vivo-expanded Treg cell population exhibits a suppressive function, wherein the suppressive function is at least 50%, at least 75%, at least 100%, or at least 150% that of baseline Tregs, as determined by suppression of proliferation of responder T cells by flow cytometry or thymidine incorporation. In some embodiments, the ex vivo-expanded Tregs are autologous to a human subject with ALS. In some embodiments, the ex vivo-expanded Tregs have been expanded from a cell sample from a human subject with ALS.

In another aspect, provided herein is a pharmaceutical composition comprising the ex vivo-expanded Treg cell population provided herein, following thawing and without further expansion, and a pharmaceutically acceptable carrier.

In some embodiments, the ability of the ex vivo-expanded Tregs to suppress inflammatory cells, following expansion and without additional expansion, is at least 70%, that of the ex vivo-expanded Tregs prior to cryopreservation. In some embodiments, the ability of the ex vivo-expanded Tregs to suppress inflammatory cells is measured by IL-6, TNFα, IL1(3, IL8, and/or Interferon-γ production by the inflammatory cells. In some embodiments, the ability to suppress inflammatory cells is measured by IL-6 production by the inflammatory cells.

In some embodiments, the therapeutic population of ex vivo-expanded Tregs, following thawing and without additional expansion, exhibits a suppressive function that is at least 50%, at least 60%, at least 70%, at least 75%, at least 80%, at least 85%, at least 90% or at least 95% as determined by suppression of proliferation of responder T cells by flow cytometry or thymidine incorporation. In some embodiments, the therapeutic population of ex vivo-expanded Tregs, following thawing and without additional expansion, exhibits a suppressive function that is at least 50%, at least 60%, at least 70%, at least 75%, at least 80%, at least 85%, at least 90% or at least 95% of the suppressive function of the ex vivo-expanded Tregs prior to cryopreservation, as determined by suppression of proliferation of responder T cells by flow cytometry or thymidine incorporation. In some embodiments, the therapeutic population of ex vivo-expanded Tregs, following thawing and without additional expansion, exhibits at least 70%, at least 75%, at least 80%, at least 85%, at least 90% or at least 95% viability, as determined by trypan blue staining.

In some embodiments, the therapeutic population of ex vivo-expanded Tregs, following thawing and without additional expansion, exhibits a viability that is at least 70%, at least 75%, at least 80%, at least 85%, at least 90% or at least 95% of the viability of the Tregs before cryopreservation, as determined by trypan blue staining.

In some embodiments, the therapeutic population of ex vivo-expanded Tregs, following thawing and without additional expansion, comprises at least 40%, at least 50%, at least 60%, at least 70%, or at least 80% or at least 90% FoxP3+ Tregs, as determined by flow cytometry. In some embodiments, the therapeutic population of ex vivo-expanded Tregs, following thawing and without additional expansion, comprises at least 70%, at least 75%, at least 80%, at least 85%, at least 90% or at least 95% CD4⁺CD25+ cells, as determined by flow cytometry. In some embodiments, the therapeutic population of ex vivo-expanded Tregs, following thawing and without additional expansion, comprises fewer than 20% CD8+ cells, as determined by flow cytometry. In some embodiments, the therapeutic population of ex vivo-expanded Tregs, following thawing and without additional expansion, comprises at least 70%, at least 80%, or at least 90% CD4⁺CD25^{high}CD127^{low} Tregs, as determined by flow cytometry. In some embodiments, the ex vivo-expanded Tregs are autologous to a human subject with ALS. In some embodiments, the ex vivo-expanded Tregs have been expanded from a cell sample from a human subject with ALS. In some embodiments, gene product expression is determined by single-shot proteomic analysis.

In another aspect, provided herein is a pharmaceutical composition comprising the cryopreserved composition comprising a therapeutic population of ex vivo-expanded Tregs provided herein following thawing and without further expansion, and a pharmaceutically acceptable carrier.

In another aspect, provided herein is a method of treating a disorder associated with Treg dysfunction, the method comprising: administering to a subject in need of said treatment a pharmaceutical composition comprising a therapeutic population of Tregs, wherein the Tregs had been ex vivo expanded and cryopreserved, and wherein the Tregs are not further expanded prior to the administering.

In another aspect, provided herein is a method of treating a disorder associated with Treg deficiency, the method comprising: administering to a subject in need of said treatment a pharmaceutical composition comprising a therapeutic population of Tregs, wherein the Tregs had been ex vivo expanded and cryopreserved, and wherein the Tregs are not further expanded prior to the administering.

In another aspect, provided herein is a method of treating a disorder associated with overactivation of the immune system, the method comprising: administering to a subject in need of said treatment a pharmaceutical composition comprising a therapeutic population of Tregs, wherein the Tregs had been ex vivo expanded and cryopreserved, and wherein the Tregs are not further expanded prior to the administering.

In another aspect, provided herein is a method of treating an inflammatory condition driven by a T cell response, the method comprising: administering to a subject in need of said treatment a pharmaceutical composition comprising a therapeutic population of Tregs, wherein the Tregs had been ex vivo expanded and cryopreserved, and wherein the Tregs are not further expanded prior to the administering.

In another aspect, provided herein is a method of treating an inflammatory condition driven by a myeloid cell response, the method comprising: administering to a subject in need of said treatment a pharmaceutical composition comprising a therapeutic population of Tregs, wherein the Tregs had been ex vivo expanded and cryopreserved, and wherein the Tregs are not further expanded prior to the administering. In some embodiments, the myeloid cell is a monocyte, macrophage or microglia.

In another aspect, provided herein is a method of treating a neurodegenerative disorder in a subject in need thereof, the method comprising: administering to the subject a pharmaceutical composition comprising a therapeutic population of Tregs, wherein the Tregs had been ex vivo expanded and cryopreserved, and wherein the Tregs are not further expanded prior to the administering. In some embodiments, the neurodegenerative disease is Amyotrophic Lateral Sclerosis (ALS), Alzheimer's disease, Parkinson's disease, frontotemporal dementia or Huntington's disease.

In another aspect, provided herein is a method of treating an autoimmune disorder in a subject in need thereof, the method comprising: administering to the subject a pharmaceutical composition comprising a therapeutic population of Tregs, wherein the Tregs had been ex vivo expanded and cryopreserved, and wherein the Tregs are not further expanded prior to the administering. In some embodiments, the autoimmune disorder is polymyositis, ulcerative colitis, inflammatory bowel disease, Crohn's disease, celiac disease, systemic sclerosis (scleroderma), multiple sclerosis (MS), rheumatoid arthritis (RA), Type I diabetes, psoriasis, dermatomyosititis, lupus, e.g., systemic lupus erythematosus, or cutaneous lupus, myasthenia gravis, autoimmune nephropathy, autoimmune hemolytic anemia, autoimmune cytopenia, autoimmune encephalitis, autoimmune hepatitis, autoimmune uveitis, alopecia, thyroiditis or pemphigus.

In another aspect, provided herein is a method of treating graft-versus-host disease in a subject in need thereof, the method comprising: administering to the subject a pharmaceutical composition comprising a therapeutic population of Tregs, wherein the Tregs had been ex vivo expanded and cryopreserved, and wherein the Tregs are not further expanded prior to the administering. In some embodiments, the subject has received a bone marrow transplant, kidney transplant or liver transplant.

In another aspect, provided herein is a method of improving islet graft survival in a subject in need thereof, comprising: combining islet transplantation with administering to the subject a pharmaceutical composition comprising a therapeutic population of Tregs, wherein the Tregs had been ex vivo expanded and cryopreserved, and wherein the Tregs are not further expanded prior to the administering.

In another aspect, provided herein is a method of treating cardio-inflammation in a subject in need thereof, the method comprising: administering to the subject a pharmaceutical composition comprising a therapeutic population of Tregs, wherein the Tregs had been ex vivo expanded and cryopreserved, and wherein the Tregs are not further expanded prior to the administering. In some embodiments, the cardio-inflammation is associated with atherosclerosis, myocardial infarction, ischemic cardiomyopathy or heart failure.

In another aspect, provided herein is a method of treating neuroinflammation in a subject in need thereof, the method comprising: administering to the subject a pharmaceutical composition comprising a therapeutic population of Tregs, wherein the Tregs had been ex vivo expanded and cryopreserved, and wherein the Tregs are not further expanded prior to the administering. In some embodiments, the neuroinflammation is associated with stroke, acute disseminated encephalomyelitis, acute optic neuritis, acute inflammatory demyelinating polyradiculoneuropathy, chronic inflammatory demyelinating polyradiculoneuropathy, Guillain-Barre syndrome, transverse myelitis, neuromyelitis optica, epilepsy, traumatic brain injury, spinal cord injury, encephalitis, central nervous system vasculitis, neurosarcoidosis, autoimmune or post-infectious encephalitis or chronic meningitis.

In another aspect, provided herein is a method of treating a Tregopathy in a subject in need thereof, comprising: administering to the subject a pharmaceutical composition comprising a therapeutic population of Tregs, wherein the Tregs had been ex vivo expanded and cryopreserved, and wherein the Tregs are not further expanded prior to the administering. In some embodiments, the Tregopathy is caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

In some embodiments of the methods of treatment provided herein, the Tregs are autologous to the subject. In other embodiments of the methods of treatment provided herein, the Tregs are allogeneic to the subject.

In some embodiments of the methods of treatment provided herein, the composition is a pharmaceutical composition provided herein.

For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

### 5. BRIEF DESCRIPTION OF THE FIGURES

FIG. 1 shows a flow chart of a process of producing a therapeutic population of Tregs in a bioreactor.

FIG. 2 is a graph showing Treg cell number versus incubation time in bioreactors for 6 Treg expansion runs.

FIG. 3 shows the viability of cryopreserved Tregs in samples at baseline, pre-freeze, and post-thaw from the 6 runs.

FIG. 4 shows the percentage of CD4⁺CD25+ Treg cells in baseline and post-thaw samples from the 6 runs.

FIG. 5 shows the percentage of CD4⁺CD25+FOXP3+ Treg cells in baseline and post-thaw samples from the 6 runs.

FIG. 6 shows the percentage of CD4⁺CD25+CD127+IoFOXP3+ Treg cells in baseline and post-thaw samples from the 6 runs.

FIG. 7 shows the suppressive function of cryopreserved Treg cells in baseline and post-thaw Treg samples.

FIG. 8 shows the protein levels of FoxP3 in cryopreserved Treg cells in baseline and post-thaw Treg samples.

FIG. 9 shows the protein levels of CD25 in cryopreserved Treg cells in baseline and post-thaw Treg samples.

### 6. ILLUSTRATIVE EMBODIMENTS

Illustrative embodiments of the invention are included in the text below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and/or time-consuming, but would be a routine undertaking for those of ordinary skill in the art having the benefit of this disclosure.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b), and wherein step (b) takes place in a bioreactor; and (c) cryopreserving the expanded Treg cell population to produce a cryopreserved therapeutic population of Tregs, wherein the enriching step begins within about 30 to 90 minutes of obtaining the leukapheresis sample, and wherein the expansion step begins within about 30-90 minutes of completion of the enrichment step.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b), and wherein step (b) takes place in a bioreactor; and (c) cryopreserving the expanded Treg cell population to produce a cryopreserved therapeutic population of Tregs, wherein the enriching step begins within about 30 to 90 minutes of obtaining the leukapheresis sample, wherein the expansion step begins within about 30-90 minutes of completion of the enrichment step, and wherein the cryopreservation step is initiated after about 15-25 days of expansion.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b), and wherein step (b) takes place in a bioreactor; and (c) cryopreserving the expanded Treg cell population to produce a cryopreserved therapeutic population of Tregs, wherein the enriching step begins within about 30 to 90 minutes of obtaining the leukapheresis sample, and wherein the expansion step (i) begins within about 30-90 minutes of completion of the enrichment step, (ii) comprises culturing the Tregs in a culture medium that comprises beads coated with anti-CD3 antibodies and anti-CD28 antibodies, and (iii) comprises the addition of an expansion agent to the culture medium every 2-3 days.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b), and wherein step (b) takes place in a bioreactor; and (c) cryopreserving the expanded Treg cell population to produce a cryopreserved therapeutic population of Tregs, wherein the enriching step begins within about 30 to 90 minutes of obtaining the leukapheresis sample, and wherein the expansion step (i) begins within about 30-90 minutes of completion of the enrichment step, (ii) comprises adding beads coated with anti-CD3 antibodies and anti-CD28 antibodies to the cell culture medium within 24 h of initiating the culturing and (iii) comprises the addition of an expansion agent to the culture medium every 2-3 days.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b), and wherein step (b) takes place in a bioreactor; and (c) cryopreserving the expanded Treg cell population to produce a cryopreserved therapeutic population of Tregs, wherein the enriching step begins within about 30 to 90 minutes of obtaining the leukapheresis sample, and wherein the expansion step (i) begins within about 30-90 minutes of completion of the enrichment step, (ii) comprises adding beads coated with anti-CD3 antibodies and anti-CD28 antibodies to the cell culture medium within about 24 h of initiating the culturing and (iii) comprises the addition of an expansion agent to the culture medium every 2-3 days, beginning within about 6 days of initiating the culturing.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b), and wherein step (b) takes place in a bioreactor; and (c) cryopreserving the expanded Treg cell population to produce a cryopreserved therapeutic population of Tregs, wherein the enriching step begins within about 30 to 90 minutes of obtaining the leukapheresis sample, and wherein the expansion step (i) begins within about 30-90 minutes of completion of the enrichment step, (ii) comprises adding beads coated with anti-CD3 antibodies and anti-CD28 antibodies to the cell culture medium within about 24 h of initiating the culturing and (iii) comprises the addition of an expansion agent to the culture medium every 2-3 days, beginning within about 6 days of initiating the culturing.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b), and wherein step (b) takes place in a bioreactor; and (c) cryopreserving the expanded Treg cell population to produce a cryopreserved therapeutic population of Tregs, wherein the enriching step begins within about 30 to 90 minutes of obtaining the leukapheresis sample, and wherein the expansion step (i) begins within about 30-90 minutes of completion of the enrichment step, (ii) comprises adding beads coated with anti-CD3 antibodies and anti-CD28 antibodies to the cell culture medium within about 24 h of initiating the culturing and (iii) comprises the addition of IL-2 to the culture medium every 2-3 days, beginning within about 6 days of initiating the culturing.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b), and wherein step (b) takes place in a bioreactor; and (c) cryopreserving the expanded Treg cell population to produce a cryopreserved therapeutic population of Tregs, wherein the enriching step begins within about 30 to 90 minutes of obtaining the leukapheresis sample, and wherein the expansion step (i) begins within about 30-90 minutes of completion of the enrichment step, (ii) comprises adding beads coated with anti-CD3 antibodies and anti-CD28 antibodies to the cell culture medium within about 24 h of initiating the culturing, (iii) comprises the addition of an expansion agent to the culture medium every 2-3 days, beginning within about 6 days of initiating the culturing, and (iv) adding rapamycin to the culture medium every 2-3 days, beginning within about 24 hours of the initiation of the culturing.

In one embodiment, provided herein is a method of producing a cryopreserved therapeutic population of regulatory T cells (Tregs), said method comprising the steps of (a) enriching Tregs from a leukapheresis sample suspected of containing Tregs, to produce a baseline Treg cell population; (b) expanding the baseline Treg cell population to produce an expanded Treg cell population, wherein the baseline Treg cell population is not cryopreserved prior to the initiation of step (b), and wherein step (b) takes place in a bioreactor; and (c) cryopreserving the expanded Treg cell population to produce a cryopreserved therapeutic population of Tregs, wherein the enriching step begins within about 30 to 90 minutes of obtaining the leukapheresis sample, wherein the expansion step (i) begins within about 30-90 minutes of completion of the enrichment step, (ii) comprises adding beads coated with anti-CD3 antibodies and anti-CD28 antibodies to the cell culture medium within about 24 h of initiating the culturing, (iii) comprises the addition of an expansion agent to the culture medium every 2-3 days, beginning within about 6 days of initiating the culturing, and (iv) adding rapamycin to the culture medium every 2-3 days, beginning within about 24 hours of the initiation of the culturing, and wherein the cryopreservation step is initiated after about 15-25 days of expansion.

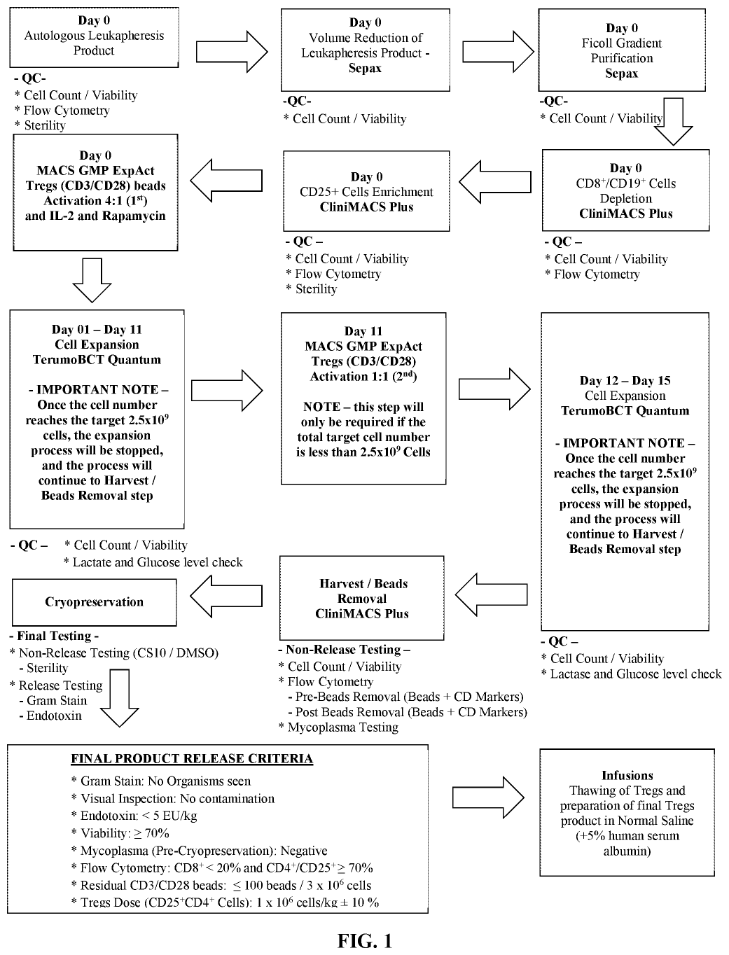

A detailed overview of an exemplary manufacturing process described herein is depicted in FIG. 1. When isolating Tregs for clinical therapies, a CD8/CD19 depletion step followed by a CD25 enrichment step is generally employed and subsequent ex vivo expansion in the presence of IL-2, which supports the survival and proliferation of Tregs, and rapamycin, which stabilizes the Treg population. This isolation and expansion strategy reduces the amount of pro-inflammatory populations (i.e., cytotoxic CDS+ T cells and T effector cells), and increases the purity of the final Treg product. The robust manufacturing of Tregs is limited by the low number of circulating Tregs that can be isolated through leukapheresis and ex vivo separation. Hence, extensive ex vivo expansion is essential in order to obtain sufficient numbers of Tregs to treat patients for a longer period of time. The expansion of functionally compromised Tregs, for example, autologous ALS-derived Tregs, is even more challenging.

Amyotrophic lateral sclerosis, also known as Lou Gehrig's disease, is a rapidly progressive and fatal neurodegenerative disease characterized by the relentless degeneration of upper and lower motor neurons. Increasing evidence shows that dysregulation of the immune system can hasten ALS disease progression. In particular, Tregs are reduced in patients with ALS and more marked reduction is associated with more rapid disease progression. Tregs are a subpopulation of T-lymphocytes consisting of CD4⁺CD25^{hi}hFOXP3+ cells that suppress neuroinflammatory responses. The safety and therapeutic potential of the adoptive transfer of autologous Tregs as a treatment for ALS has been demonstrated in a Phase I clinical study. In order to complete the phase 2 trial, in which a larger number of ALS patients will be treated with monthly doses of Tregs over one year, the manufacturing of at least 2 billion and preferably at least 2.5 billion Tregs from each study participant is required to meet the dosing demands. Similar demands will be required of an approved therapeutic regimen.

The Treg manufacturing processes described herein yield robust Treg expansion and non-deleterious cryopreservation. Current Treg manufacturing protocols are complicated and labor-intensive, which drive up manufacturing costs and are not sustainable as a therapy for large numbers of patients, for example patients with neurodegenerative diseases such as ALS or Alzheimer's disease. The bioreactor-based methods described herein address manufacturing challenges at a lower cost through optimization of the manufacturing process, e.g., cGMP manufacturing processes. Moreover, the methods described herein produce an enhanced Treg product with superior suppressive functions that are maintained even when the Tregs are cryopreserved and thawed without further expansion, wherein the Tregs could be more efficacious when infused back into the patients.

For example, the improved Treg manufacturing processes described herein are critical to the advancement of developing a therapy that drastically slows disease progression in ALS because it provides a platform for current and future clinical studies in ALS, and for therapeutic ALS regimens, allows for the effective cryopreservation of ALS-derived autologous Tregs for extended treatment times with successive doses, generates functionally superior Treg products, and is potentially an “off-the-shelf immune-privileged Treg therapy that can be used for treatment of diseases, for example, neurodegenerative diseases including ALS and Alzheimer's disease, autoimmune diseases including Type 1 diabetes and rheumatoid arthritis, and graft versus host disease (GVHD) including GVHD following bone marrow transplantation.

Regulatory T cells (Tregs) account for 5-10% of CD4+ T cells in the peripheral circulation. Their dysfunction contributes to the rapid progression of amyotrophic lateral sclerosis (ALS). The suppressive functions of Tregs isolated from patients with ALS normalize following ex vivo expansion with interleukin (IL)-2 and rapamycin. Thus, expanded functional Tregs represent a potential treatment for ALS that could slow the rate of progression. However, the particular susceptibility of the Treg population to the ongoing disease process and the autologous nature of the proposed treatment pose significant challenges to the development of a Treg therapy that could combat ALS in potentially thousands of patients. A sensible Treg manufacturing process for ALS patients requires an expansion phase that yields sufficient numbers of highly suppressive Tregs to avoid exposing the patients to frequent leukapheresis procedures for Treg isolation. Frequent infusions of optimized Treg doses would be required to continually suppress the progressive neuroinflammatory environment that ensues as ALS progresses. As it would be impractical to expand Tregs from each ALS patient prior to each infusion, the development of a cryopreservation process is crucial in order to limit the per-patient costs of cell manufacturing as well as the manpower that would be needed to develop Treg therapies for potentially thousands of patients with ALS. The Treg manufacturing processes described herein have been optimized to produce and cryopreserve large numbers of highly suppressive Tregs for their application in treatment regimens, for example, for their application in methods of treating disorders such as, e.g., neurodegenerative disorders such as ALS and Alzheimer's disease, autoimmune disorders such as Type 1 diabetes and rheumatoid arthritis, and graft versus host disease (GVHD) such as following a bone marrow transplantation, as well as for their application in future clinical trials for ALS patients and potentially other neurodegenerative diseases such as Alzheimer's disease. An optimized Treg therapy minimizes the number of expansion phases and infusions, which makes the therapy more cost effective and sustainable.

Further Illustrative embodiments are as follows:
1. A method of producing a cryopreserved therapeutic population of regulatory T cells (Tregs), said method comprising the steps of:
   a. enriching Tregs from a cell sample suspected of containing Tregs, to produce a baseline Treg cell population;
   b. expanding the baseline Treg cell population to produce an expanded Treg cell population, wherein the baseline Treg cell population is not cryopreserved prior to the initiation of step (b), and wherein step (b) takes place in a bioreactor; and
   c. cryopreserving the expanded Treg cell population to produce a cryopreserved therapeutic population of Tregs.
2. The method of embodiment 1, wherein step (a) comprises depleting CD8+/CD19+ cells then enriching for CD25+ cells.
3. The method of embodiment 1 or 2, wherein step (b) is carried out within about 30 minutes after step (a).
4. The method of any one of embodiments 1-3, wherein step (b) comprises culturing the Tregs in a culture medium that comprises beads coated with anti-CD3 antibodies and anti-CD28 antibodies.
5. The method of embodiment 4, wherein step (b) comprises adding the beads to the culture medium within about 24 hours of the initiation of the culturing.
6. The method of embodiment 4 or 5, wherein step (b) comprises adding the beads to the culture medium within about 30 minutes after the completion of step (a).
7. The method of any one of embodiments 4-6, wherein step (b) comprises adding beads coated with anti-CD3 antibodies and anti-CD28 antibodies to the culture medium about 11 days after beads coated with anti-CD3 antibodies and anti-CD28 antibodies are first added to the culture medium.
8. The method of embodiment 7, wherein step (b) comprises adding beads coated with anti-CD3 antibodies and anti-CD28 antibodies to the culture medium about 11 days after beads coated with anti-CD3 antibodies and anti-CD28 antibodies are first added to the culture medium, if the cell number by then has not reached a target cell number.
9. The method of embodiment 8, wherein the target cell number is 2.5×10⁹ cells.
10. The method of any one of embodiments 1-9, wherein step (b) comprises culturing the Tregs in a culture medium that comprises Interleukin-2 (IL-2).
11. The method of embodiment 10, wherein step (b) comprises adding IL-2 to the culture medium within about 24 hours of the initiation of the culturing.
12. The method of embodiment 10 or 11, wherein step (b) comprises adding IL-2 to the culture medium within about 30 minutes after the completion of step (a).
13. The method of any one of embodiments 10-12, wherein step (b) comprises replenishing the culture medium with IL-2 about every 3-4 days after IL-2 is first added to the culture medium.
14. The method of any one of embodiments 10-13, wherein step (b) comprises adjusting IL-2 concentration depending on cell number.
15. The method of embodiment 14, wherein step (b) comprises culturing the Tregs in a culture medium containing about 200 IU/mL IL-2 until the cell number reaches 600×10⁶, and then culturing the Tregs in a culture medium containing about 250 IU/mL IL-2.
16. The method of any one of embodiments 1-15, wherein step (b) comprises culturing the Tregs in a culture medium that comprises rapamycin.
17. The method of embodiment 16, wherein step (b) comprises adding rapamycin to the culture medium within about 24 hours of the initiation of the culturing.
18. The method of embodiment 16 or 17, where step (b) comprises adding rapamycin to the culture medium within about 30 minutes after the completion of step (a).
19. The method of any one of embodiments 1-18, wherein step (b) comprises adjusting flow rate of an extracapillary (EC) medium of the bioreactor depending on cell number.
20. The method of embodiment 19, wherein the extracapillary medium comprises rapamycin.
21. The method of embodiment 19 or 20, wherein step (b) comprises maintaining the flow rate of the EC medium at 0 until the cell number reaches 500×10⁶, then increasing the flow rate of the EC medium to about 0.2 mL/min and maintaining the flow rate of the EC medium at about 0.2 mL/min until the cell number reaches 750×10⁶, then increasing the flow rate of the EC medium to about 0.4 mL/min and maintaining the flow rate of the EC medium at about 0.4 mL/min until the cell number reaches about 1,000×10⁶, then increasing the flow rate of the EC medium to about 0.6 mL/min and maintaining the flow rate of the EC medium at about 0.6 mL/min until the cell number reaches about 1,500×10⁶, and then increasing the flow rate of the EC medium to about 0.8 mL/min and maintaining the flow rate of the EC medium at about 0.8 mL/min.
22. The method of any one of embodiments 1-21, wherein the cell sample is a leukapheresis cell sample.
23. The method of any one of embodiments 1-22, wherein step (b) is automated.
24. The method of any one of embodiments 1-23, wherein step (b) takes place in a closed system.
25. The method of any one of embodiments 1-24, wherein step (a) is automated.
26. The method of any one of embodiments 1-25, wherein step (a) takes place in a closed system.
27. The method of any one of embodiments 1-26, wherein step (a) and step (b) take place in different systems.
28. The method of embodiment 27, wherein the baseline Treg cell population produced by step (a) are transferred to the bioreactor in step (b) in a closed step.
29. The method of embodiment 27, wherein step (a) takes place in a closed system, step (b) takes place in a closed system, and the baseline Treg cell population produced by step (a) are transferred to the bioreactor in step (b) in a closed step.
30. The method of any one of embodiments 1-26, wherein step (a) and step (b) take place in the same system.
31. The method of embodiment 30, wherein the same system is a closed system.
32. The method of any one of embodiments 1-31, wherein the method further comprises thawing the cryopreserved therapeutic population of Tregs and, without further expansion, placing the population into a composition comprising a pharmaceutically acceptable carrier, to produce a pharmaceutical composition.
33. The method of embodiment 32, wherein the method further comprises administering the pharmaceutical composition to a subject.
34. The method of embodiment 33, wherein the Tregs in the pharmaceutical composition are autologous to the subject.
35. The method of embodiment 33, wherein the Tregs in the pharmaceutical composition are allogeneic to the subject.
36. The method of any one of embodiments 33-35, wherein the subject is a human subject.
37. A cryopreserved therapeutic population of Tregs produced by the method of any one of embodiments 1-31.
38. A pharmaceutical composition comprising a thawed and unexpanded form of the cryopreserved therapeutic population of Tregs of embodiment 37, and a pharmaceutically acceptable carrier.
39. A pharmaceutical composition produced by the method of any one of embodiments 32-36.
40. A method of treating a disorder associated with Treg dysfunction in a subject in need thereof, the method comprising administering to the subject the pharmaceutical composition of embodiment 38 or 39.
41. A method of treating a disorder associated with Treg deficiency in a subject in need thereof, the method comprising administering to the subject the pharmaceutical composition of embodiment 38 or 39.
42. A method of treating a disorder associated with overactivation of the immune system in a subject in need thereof, the method comprising administering to the subject the pharmaceutical composition of embodiment 38 or 39.
43. A method of treating an inflammatory condition driven by a T cell response in a subject in need thereof, the method comprising administering to the subject the pharmaceutical composition of embodiment 38 or 39.
44. A method of treating an inflammatory condition driven by a myeloid cell response in a subject in need thereof, the method comprising administering to the subject the pharmaceutical composition of embodiment 38 or 39.
45. The method of embodiment 44, wherein the myeloid cell is a monocyte, macrophage or microglia.
46. A method of treating a neurodegenerative disorder in a subject in need thereof, the method comprising administering to the subject the pharmaceutical composition of embodiment 38 or 39.
47. The method of embodiment 46, wherein the neurodegenerative disease is Amyotrophic Lateral Sclerosis (ALS), Alzheimer's disease, Parkinson's disease, frontotemporal dementia or Huntington's disease.
48. A method of treating an autoimmune disorder in a subject in need thereof, the method comprising administering to the subject the pharmaceutical composition of embodiment 38 or 39.
49. The method of embodiment 48, wherein the autoimmune disorder is polymyositis, ulcerative colitis, inflammatory bowel disease, Crohn's disease, celiac disease, systemic sclerosis (scleroderma), multiple sclerosis (MS), rheumatoid arthritis (RA), Type I diabetes, psoriasis, dermatomyosititis, lupus, e.g., systemic lupus erythematosus, or cutaneous lupus, myasthenia gravis, autoimmune nephropathy, autoimmune hemolytic anemia, autoimmune cytopenia, autoimmune encephalitis, autoimmune hepatitis, autoimmune uveitis, alopecia, thyroiditis or pemphigus.
50. A method of treating graft-versus-host disease in a subject in need thereof, the method comprising administering to the subject the pharmaceutical composition of embodiment 38 or 39.
51. The method of embodiment 50, wherein the subject has received a bone marrow transplant, kidney transplant or liver transplant.
52. A method of improving islet graft survival in a subject in need thereof, comprising administering to the subject the pharmaceutical composition of embodiment 38 or 39 in combination with the islet transplantation.
53. A method of treating cardio-inflammation in a subject in need thereof, the method comprising administering to the subject the pharmaceutical composition of embodiment 38 or 39.
54. The method of embodiment 53, wherein the cardio-inflammation is associated with atherosclerosis, myocardial infarction, ischemic cardiomyopathy or heart failure.
55. A method of treating neuroinflammation in a subject in need thereof, the method comprising administering to the subject the pharmaceutical composition of embodiment 38 or 39.
56. The method of embodiment 55, wherein the neuroinflammation is associated with stroke, acute disseminated encephalomyelitis, acute optic neuritis, acute inflammatory demyelinating polyradiculoneuropathy, chronic inflammatory demyelinating polyradiculoneuropathy, Guillain-Barre syndrome, transverse myelitis, neuromyelitis optica, epilepsy, traumatic brain injury, spinal cord injury, encephalitis, central nervous system vasculitis, neurosarcoidosis, autoimmune or post-infectious encephalitis or chronic meningitis.
57. A method of treating a Tregopathy in a subject in need thereof, comprising administering to the subject the pharmaceutical composition of embodiment 38 or 39.
58. The method of embodiment 57, wherein the Tregopathy is caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

### 7. DETAILED DESCRIPTION

Presented herein are bioreactor-based methods of producing ex vivo-expanded Treg cell populations, compositions comprising such ex vivo-expanded Treg cell populations, including pharmaceutical compositions comprising such Treg cell populations, cryopreserved ex vivo-expanded therapeutic Treg populations, and pharmaceutical compositions comprising such cryopreserved Tregs following their thawing and without further expansion.

Also presented herein are methods of treatment that utilize the Treg cell populations produced and described herein, including, for example, treatment of neurodegenerative disorders such as amyotrophic lateral sclerosis (ALS), Alzheimer's disease, Parkinson's disease and frontotemporal dementia.

Recitation of ranges of values herein are merely intended to serve as a shorthand method of referring individually to each separate value falling within the range, unless otherwise indicated herein, and each separate value is incorporated into the specification as if it were individually recited herein.

Unless specifically stated or apparent from context, as used herein, the terms “a”, “an”, and “the” are understood to be singular or plural, and denote “one or more.”

The terms “include,” “such as,” and the like are intended to convey inclusion without limitation, unless otherwise specifically indicated.

The terms “or” and “and” can be used interchangeably and can be understood to mean “and/or.”

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range. In particular, unless otherwise noted the terms mean within plus or minus 10% of a given value or range. In instances where an integer is required, the terms mean within plus or minus 10% of a given value or range, rounded either up or down to the nearest integer.

### 7.1 Methods of Producing Expanded Therapeutic Populations of Tregs

Provided herein are methods for the production of expanded Treg cell populations, cryopreserved therapeutic populations of Tregs, and pharmaceutical compositions comprising cryopreserved expanded Tregs that have been thawed and placed into compositions comprising a pharmaceutically acceptable carrier without further expansion, particularly for use in connection with treating neurodegenerative diseases, autoimmune diseases and other diseases with an inflammatory component. In some embodiments, the method involves the growth and manipulation of patient Tregs outside of the body.

The methods for the production of therapeutic populations of Tregs provided herein may be useful for treating patients with a pathological disease or condition. Also provided herein are therapeutic populations of Tregs produced by methods described herein and pharmaceutical compositions thereof.

In some embodiments, the therapeutic populations of Tregs produced by a method provided herein have advantageous properties for clinical application. For example, in one embodiment, a therapeutic population of Tregs produced by a method provided herein may be cryopreserved without loss of viability, purity or potency. For example, in one embodiment, a therapeutic population of ex vivo-expanded Tregs produced by a method provided herein may be cryopreserved, thawed and without further expansion, demonstrate maintenance of viability, purity and potency as compared to the expanded Tregs prior to cryopreservation. In another embodiment, a therapeutic population of Tregs produced by a method described herein comprises Tregs with higher suppressive ability than the Tregs enriched from the donor samples, or compared to a healthy donor's Tregs. In yet another embodiment, a therapeutic population of Tregs produced by a method described herein comprises Tregs with a suppressive ability that is absent Tregs enriched from the donor samples, or compared to a healthy donor's Tregs. Thus, in some embodiments, a method of producing a therapeutic population of Tregs provided herein is an improved method compared to methods known in the art.

In some embodiments, the method of producing a therapeutic population of Tregs comprises the steps of (1) enriching a cell population obtained from a subject for Tregs; (2) ex vivo expansion of the cell population enriched for Tregs and/or (3) cryopreservation of the expanded Tregs. A population of cells comprising Tregs may be enriched from a biological sample, e.g., a peripheral blood sample or thymic tissue. In certain embodiments, step (2) is automated. In certain embodiments, step (2) takes place in a closed system. In certain embodiments, step (2) is automated and takes place in a closed system. In specific embodiments, step (2) takes place in a bioreactor (e.g., a Terumo BCT Quantum® Cell Expansion System). In certain embodiments, step (1) is automated. In certain embodiments, step (1) takes place in a closed system. In certain embodiments, step (1) is automated and takes place in a closed system. In specific embodiments, step (1) takes place in a CliniMACS Prodigy® system. In specific embodiments, step (1) takes place in a CliniMACS® Plus system. In some embodiments, step (1) and step (2) take place in different systems (for example, step (1) takes place in a CliniMACS Prodigy® system and step (2) takes place in a Terumo BCT Quantum® Cell Expansion System, or step (1) takes place in a CliniMACS® Plus system and step (2) takes place in a Terumo BCT Quantum® Cell Expansion System). In specific embodiments, the enriched cell population produced by step (1) are transferred to the system where step (2) takes place in a closed step. In other embodiments, step (1) and step (2) take place in the same system. In specific embodiments, the same system is a closed system.

In one aspect, provide herein is a method of producing a cryopreserved therapeutic population of regulatory T cells (Tregs), said method comprising the steps of. (a) enriching Tregs from a cell sample suspected of containing Tregs, to produce a baseline Treg cell population; (b) expanding the baseline Treg cell population to produce an expanded Treg cell population, wherein the baseline Treg cell population is not cryopreserved prior to the initiation of step (b), and wherein step (b) takes place in a bioreactor; and (c) cryopreserving the expanded Treg cell population to produce a cryopreserved therapeutic population of Tregs. In this context, the term “baseline,” or “baseline Treg cell population” denotes a population of Tregs that has been enriched from a patient sample but has not yet been expanded. In certain embodiments, step (b) is automated. In certain embodiments, step (b) takes place in a closed system. In certain embodiments, step (b) is automated and takes place in a closed system. In specific embodiments, step (b) takes place in a Terumo BCT Quantum® Cell Expansion System. In certain embodiments, step (a) is automated. In certain embodiments, step (a) takes place in a closed system. In certain embodiments, step (a) is automated and takes place in a closed system. In specific embodiments, step (a) takes place in a CliniMACS Prodigy® system. In specific embodiments, step (a) takes place in a CliniMACS® Plus system. In some embodiments, step (a) and step (b) take place in different systems (for example, step (a) takes place in a CliniMACS Prodigy® system and step (b) takes place in a Terumo BCT Quantum® Cell Expansion System, or step (a) takes place in a CliniMACS® Plus system and step (b) takes place in a Terumo BCT Quantum® Cell Expansion System). In specific embodiments, the baseline Treg cell population produced by step (a) are transferred to the bioreactor in step (b) in a closed step. In other embodiments, step (a) and step (b) take place in the same system. In specific embodiments, the same system is a closed system.

### 7.1.1. Treg Enrichment

In some embodiments, methods of producing a therapeutic population of Tregs provided herein comprise a step of enriching Tregs from in a biological donor sample, e.g., a peripheral blood sample or thymic tissue. In some embodiments, the therapeutic population of Tregs is obtained from a serum sample suspected of containing Tregs. In some embodiments, the therapeutic population of Tregs is obtained from a cell sample suspected of containing Tregs, obtained from a donor via leukapheresis. In some embodiments, the therapeutic population of Tregs is obtained from a biological sample suspected of containing Tregs. It shall be understood that methods of producing a therapeutic population of Tregs provided or described herein include methods of producing a cryopreserved therapeutic population of Tregs.

In some embodiments, the therapeutic population of Tregs is enriched from a biological sample from a donor subject, in particular a human donor subject. In some embodiments, the donor of the biological sample is the patient subject to be treated by the therapeutic population of Tregs or a derivative thereof. In other embodiments, the donor of the biological sample is different from the patient subject to be treated by the therapeutic population of Tregs or a derivative thereof. The biological sample can be any sample suspected of containing Tregs, likely to contain Tregs or know to contain Tregs. Such biological samples may be taken directly from the subject, or may be samples resulting from one or more processing steps, such as separation, e.g. selection or enrichment, centrifugation, washing, and/or incubation. Biological samples include, but are not limited to, body fluids, such as blood, plasma, serum, cerebrospinal fluid, synovial fluid, tissue and organ samples, including processed samples derived therefrom.

In some aspects, the sample is blood or a blood-derived sample, or is or is derived from an apheresis or leukapheresis product. Exemplary samples include whole blood, peripheral blood mononuclear cells (PBMCs), leukocytes, bone marrow, and thymus.

In some embodiments, the biological sample is a blood-derived sample, e.g., a samples derived from whole blood, serum, or plasma. In some embodiments, the biological sample is or includes peripheral blood mononuclear cells. In some embodiments, the biological sample is a peripheral blood or serum sample. In some embodiments, the biological sample is a lymph node sample.

In some embodiments, the donor subject is a human subject. In some embodiments, the human donor is a healthy donor.

In some embodiments, the donor subject is diagnosed with or is suspected of having a disorder associated with Treg dysfunction. In some embodiments, the donor subject is diagnosed with or is suspected of having a disorder associated with Treg deficiency. In some embodiments, the donor subject is diagnosed with or is suspected of having a condition driven by a T cell response.

In some embodiments the donor subject is diagnosed with or is suspected of having a neurodegenerative disease. In some embodiments, the donor subject is diagnosed with or is suspected of having Alzheimer's disease, Amyotrophic Lateral Sclerosis, Huntington's disease or frontotemporal dementia.

In some embodiments, the donor subject is diagnosed with or is suspected of having a disorder that would benefit from downregulation of the immune system.

In some embodiments, the donor subject is diagnosed with or suspected of having an autoimmune disease. The autoimmune disease may be, for example, systemic sclerosis (scleroderma), polymyositis, ulcerative colitis, inflammatory bowel disease, Crohn's disease, celiac disease, multiple sclerosis (MS), rheumatoid arthritis (RA), Type I diabetes, psoriasis, dermatomyositis, lupus, systemic lupus erythematosus, or cutaneous lupus, myasthenia gravis, autoimmune nephropathy, autoimmune hemolytic anemia, autoimmune cytopenia autoimmune hepatitis, autoimmune uveitis, alopecia, thyroiditis or pemhigus.

In some embodiments, the donor subject is diagnosed with or suspected of having heart failure or ischemic cardiomyopathy. In some embodiments, the donor subject is diagnosed with or suspected of having graft-versus-host disease, e.g., after undergoing organ transplantation (such as a kidney transplantation or a liver transplantation), or after undergoing stem cell transplantation (such as hematopoietic stem cell transplantation).

In some embodiments, the donor subject is diagnosed with or suspected of having neuroinflammation. Neuroinflammation may be associated, for example, with stroke, acute disseminated encephalitis, acute optic neuritis, transverse myelitis, neuromyelitis optica, epilepsy, traumatic brain injury, spinal cord injury, encephalitis central nervous system (CNS) vasculitis, neurosarcoidosis, autoimmune or post-infectious encephalitis or chronic meningitis.

In some embodiments, the donor subject is diagnosed with or suspected of having chronic inflammatory demyelinating polyradiculoneuropathy (CIDP). In some embodiments, the donor subject is diagnosed with or suspected of having acute inflammatory demyelinating polyneuropathy (AIDP). In some embodiments, the donor subject is diagnosed with or suspected of having Guillain-Barre syndrome (GBS).

In some embodiments, the donor subject is diagnosed with or suspected of having cardo-inflammation, e.g., cardio-inflammation associated with myocardial infarction, ischemic cardiomyopathy, with heart failure.

In some embodiments, the donor subject has had a stroke.

In some embodiments, the donor subject is diagnosed with or suspected of having cancer, e.g., a blood cancer.

In some embodiments, the donor subject is diagnosed with or suspected of having asthma.

In some embodiments, the donor subject is diagnosed with or suspected of having eczema.

In some embodiments, the donor subject is diagnosed with or suspected of having a disorder associated with overactivation of the immune system.

In some embodiments, the donor subject is diagnosed with or suspected of having Tregopathy. The Tregopathy may be caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

Methods of obtaining a population of cells suspected to contain, likely to contain or known to contain Tregs from such biological donor samples are known in the art. For example, lymphocytes may be obtained from a peripheral blood sample by leukapheresis. In some embodiments, Tregs are enriched from a population of lymphocytes. In some embodiments, repeated peripheral blood samples are obtained from a donor for producing Tregs. In some embodiments, two or more peripheral blood samples are obtained from a donor. In some embodiments, insufficient Tregs are obtained from a donor sample after expanding for 25 days and a subsequent sample is obtained. In some embodiments, the donor sample undergoes volume reduction (e.g., volume reduction by a method described herein) during the enrichment process.

In some embodiments, biological samples (e.g., leukapheresis samples) from more than one donor are pooled prior to the enrichment process to generate an allogeneic population of Tregs. In some embodiments, biological samples (e.g., leukapheresis samples from 2, 3, 4, or 5 donors are pooled.

Tregs may be enriched from a biological sample by any method known in the art or described herein. In some embodiments, Tregs are enriched from a sample using magnetic bead separation (e.g., CliniMACS Tubing Set LS (162-01), CliniMACS® Plus Instrument or CliniMACS Prodigy® Instrument), fluorescent cell sorting, and/or disposable closed cartridge based cell sorters.

Enrichment for cells expressing one or more markers refers to increasing the number or percentage of such cells in the population of cells, but does not necessarily result in a complete absence of cells not expressing the marker. Depletion of cells expressing one or more markers refers to decreasing the number or percentage of such cells in the population of cells, but does not necessarily result in a complete removal of all cells expressing such marker or markers.

In some embodiments, the enrichment comprises a step of affinity- or immunoaffinity-based separation of cells expressing one or more markers (e.g., Treg cell surface markers). Such separation steps can be based on positive selection, in which the cells expressing one or more markers are retained, and/or on negative selection (depletion), in which the cells not expressing one or more markers are retained.

The separation may be based on the expression (e.g., positive or negative expression) or expression level (e.g., high or low expression) of one or more markers (e.g., Treg cell surface markers). In this context, “high expression” and “low expression” are generally relative to the whole population of cells. In some embodiments, separation of cells may be based on CD8 expression. In some embodiments, separation of cells may be based on CD19 expression. In some embodiments, separation of cells may be based on high CD25 expression.

Thus, in some embodiments, enrichment of Tregs may comprise incubation with an antibody or binding partner that specifically binds to a marker (e.g., a Treg cell surface marker), followed generally by washing steps and separation of cells having bound the antibody or binding partner from those cells having not bound to the antibody or binding partner.

In some embodiments, the antibody or binding partner is bound to a solid support or matrix, such as a sphere or bead, for example a nanoparticle, microbeads, nanobeads, including agarose, magnetic bead or paramagnetic beads. In some embodiments, the spheres or beads can be packed into a column to effect immunoaffinity chromatography. In some embodiments, the antibody or binding partner is detectably labeled. In some embodiments, the antibody or binding partner is attached to small, magnetically responsive particles or microparticles, such as nanoparticles or paramagnetic beads. Such beads are known and are commercially available (e.g., Dynabeads® (Life Technologies, Carlsbad, CA), MACS® beads (Miltenyi Biotec, San Diego, CA) or Streptamer® bead reagents (IBA, Germany)). Such particles or microparticles may be incubated with the population of cells to be enriched and then placed in a magnetic field. This results in those cells that are attached to the particles or microparticles via the antibody or binding partner being attracted to the magnet and separated from the unbound cells. This method allows for retention of the cells attached to the magnet (positive selection) or removal of the cells attracted to the magnet (negative selection).

In some embodiments, a method of producing a therapeutic population of Tregs provided herein comprises both positive and negative selection during the enrichment step.

In some embodiments, the biological sample is obtained within about 25-35 min, about 35-45 min, about 45-60 min, about 60-75 min, about 75-90 min, about 90-120 min, about 120-150 min, about 150-180 min, about 2-3 h, about 3-4 h, about 4-5 h or about 5-6 h of the beginning of the enriching step. In some embodiments, the sample is obtained within about 30 min of the beginning of the enriching step. In some embodiments, the biological sample is not stored (e.g., stored at 4° C.) overnight.

In some embodiments, enrichment of Tregs from a human sample comprises depleting the sample of CD8+ cells. In some embodiments, enrichment of Tregs from a human sample comprises depleting a sample of CD19+ cells. In some embodiments, enrichment of Tregs from a biological sample comprises depleting the sample of CD8+ cells and CD19+ cells. In some embodiments, enrichment of Tregs from a biological sample comprises enriching the cell population for CD25^{high} cells. In some embodiments, enrichment of Tregs from a biological sample comprises enriching the cell population for CD25+ cells. In some embodiments, enrichment of Tregs from a biological sample comprises depletion of CD8+ cells and CD19+ cells from the sample and enriching the cell population for CD25^{high} cells. In some embodiments, enrichment of Tregs from a biological sample comprises depleting CD8+/CD19+cells and enriching for CD25+ cells.

In some embodiments, the population of cells enriched for Tregs comprises an increased proportion of CD4+CD25^{high} Tregs relative to the proportion of CD4+CD25^{high} Tregs in the Tregs prior to enrichment as determined by flow cytometry. In specific embodiments, the proportion of CD4+CD25^{high} Tregs is increased by about 2-fold to about 4-fold, about 4-fold to about 6-fold, about 6-fold to about 8-fold, about 8-fold to about 10-fold, about 10-fold to about 15-fold, about 15-fold to about 20-fold, about 20-fold to about 25-fold, about 25-fold to about 30-fold, about 30-fold to about 35-fold, about 35-fold to about 40-fold, about 40-fold to about 45-fold, about 45-fold to about 50-fold.

In some embodiments, the population of cells enriched for Tregs comprises an increased proportion of CD4+CD25^{high}CD127^{low} Tregs relative to the proportion of CD4+CD25^{high}CD127^{low} Tregs in the Tregs prior to enrichment as determined by flow cytometry. In specific embodiments, the proportion of CD4+CD25^{high}CD127^{low} Tregs is increased by about 2-fold to about 4-fold, about 4-fold to about 6-fold, about 6-fold to about 8-fold, about 8-fold to about 10-fold, about 10-fold to about 15-fold, about 15-fold to about 20-fold, about 20-fold to about 25-fold, about 25-fold to about 30-fold, about 30-fold to about 35-fold, about 35-fold to about 40-fold, about 40-fold to about 45-fold, about 45-fold to about 50-fold.

In some embodiments, the population of cells enriched for Tregs comprises CD25+Tregs wherein the expression of CD25 in the Tregs is increased relative to the expression of CD25 in the Tregs prior to enrichment, as determined by flow cytometry. In specific embodiments, the expression of CD25 is increased by at least about 5-fold, at least about 10-fold, at least about 15-fold, at least about 20-fold, at least about 25-fold, at least about 30-fold, at least about 35-fold, at least about 40-fold, at least about 45-fold, or at least about 50-fold.

In some embodiments, the population of cells enriched for Tregs comprises CD127+Tregs wherein the expression of CD127 in the Tregs is increased relative to the expression of CD127 in the Tregs prior to enrichment, as determined by flow cytometry. In specific embodiments, the expression of CD127 is increased by at least about 1.5-fold, at least about 2-fold, at least about 2.5-fold, or at least about 3-fold.

In some embodiments, the granularity of the Tregs in the enriched population of Tregs is increased relative to the granularity of the Tregs prior to enrichment, as determined by flow cytometry. In specific embodiments, the granularity of the Tregs increased by at least about 1.5-fold, at least about 2-fold, at least about 2.5-fold, or at least about 3-fold.

In some embodiments, the size of the Tregs in the enriched population of Tregs is increased relative to the size of the Tregs prior to enrichment, as determined by flow cytometry. In specific embodiments, the size of the Tregs increased by at least about 1.2-fold, at least about 1.5-fold, or at least about 2-fold.

### 7.1.2. Methods of Expanding Tregs Ex Vivo

In another aspect, a method provided herein comprises a step of expanding the therapeutic population of Tregs enriched from a biological sample.

In some embodiments, the expansion step is carried out within about 4-5 days after the completion of the enrichment step. In some embodiments, the expansion step is carried out within about 3-4 days after the completion of the enrichment step. In some embodiments, the expansion step is carried out within about 2-3 days after the completion of the enrichment step. In some embodiments, the expansion step is carried out within about 1-2 days after the completion of the enrichment step. In some embodiments, the expansion step is carried out within about 24 hours after the completion of the enrichment step. In some embodiments, the expansion step is carried out within about 12 hours after the completion of the enrichment step. In some embodiments, the expansion step is carried out within about 6 hours after the completion of the enrichment step. In some embodiments, the expansion step is carried out within about 3 hours after the completion of the enrichment step. In some embodiments, the expansion step is carried out within about 2 hours after the completion of the enrichment step. In some embodiments, the expansion step is carried out within about 1 hour after the completion of the enrichment step. In some embodiments, the expansion step is carried out within about 30 minutes after the completion of the enrichment step.

This expansion of the therapeutic population of Tregs may comprise culturing the cells that have been enriched from a biological samples in media, for example, in serum-free media (e.g., TexMACS Medium), in serum-depleted media, or in serum-containing media.

In certain embodiments, the expansion step comprises culturing the Tregs in a culture medium that comprises human serum (e.g., TexMACS GMP Medium supplemented with human serum). In specific embodiments, the culture medium comprises 5% or less human serum. In specific embodiments, the culture medium comprises 4% or less human serum. In specific embodiments, the culture medium comprises 3% or less human serum. In specific embodiments, the culture medium comprises 2% or less human serum. In specific embodiments, the culture medium comprises 1% or less human serum. In specific embodiments, the culture medium comprises 0.5% or less human serum. In specific embodiments, the culture medium comprises less than 5% human serum. In specific embodiments, the culture medium comprises less than 4% human serum. In specific embodiments, the culture medium comprises less than 3% human serum. In specific embodiments, the culture medium comprises less than 2% human serum. In specific embodiments, the culture medium comprises less than 1% human serum. In specific embodiments, the culture medium comprises less than 0.5% human serum. In specific embodiments, the culture medium comprises 0-0.5% human serum. In specific embodiments, the culture medium comprises 0.5-1% human serum. In specific embodiments, the culture medium comprises 1-2% human serum. In specific embodiments, the culture medium comprises 2-3% human serum. In specific embodiments, the culture medium comprises 3-4% human serum. In specific embodiments, the culture medium comprises 4-5% human serum. In a specific embodiment, the culture medium comprises about 0.5% human serum. In another specific embodiment, the culture medium comprises about 1% human serum. In another specific embodiment, the culture medium comprises about 2% human serum. In another specific embodiment, the culture medium comprises about 3% human serum. In another specific embodiment, the culture medium comprises about 4% human serum. In another specific embodiment, the culture medium comprises about 5% human serum.

In certain embodiments, the expansion step comprises culturing the Tregs in a culture medium that comprises human AB serum (e.g., TexMACS GMP Medium supplemented with human AB serum). In specific embodiments, the culture medium comprises 5% or less human AB serum. In specific embodiments, the culture medium comprises 4% or less human AB serum. In specific embodiments, the culture medium comprises 3% or less human AB serum. In specific embodiments, the culture medium comprises 2% or less human AB serum. In specific embodiments, the culture medium comprises 1% or less human AB serum. In specific embodiments, the culture medium comprises 0.5% or less human AB serum. In specific embodiments, the culture medium comprises less than 5% human AB serum. In specific embodiments, the culture medium comprises less than 4% human AB serum. In specific embodiments, the culture medium comprises less than 3% human AB serum. In specific embodiments, the culture medium comprises less than 2% human AB serum. In specific embodiments, the culture medium comprises less than 1% human AB serum. In specific embodiments, the culture medium comprises less than 0.5% human AB serum. In specific embodiments, the culture medium comprises 0-0.5% human AB serum. In specific embodiments, the culture medium comprises 0.5-1% human AB serum. In specific embodiments, the culture medium comprises 1-2% human AB serum. In specific embodiments, the culture medium comprises 2-3% human AB serum. In specific embodiments, the culture medium comprises 3-4% human AB serum. In specific embodiments, the culture medium comprises 4-5% human AB serum. In a specific embodiment, the culture medium comprises about 0.5% human AB serum. In another specific embodiment, the culture medium comprises about 1% human AB serum. In another specific embodiment, the culture medium comprises about 2% human AB serum. In another specific embodiment, the culture medium comprises about 3% human AB serum. In another specific embodiment, the culture medium comprises about 4% human AB serum. In another specific embodiment, the culture medium comprises about 5% human AB serum.

In some embodiments, the cells enriched from a biological sample are cultured about 37° C. and about 5% C02. In some embodiments, the cells enriched from a biological sample are cultured out under good manufacturing practice (GMP) conditions. In some embodiments, the cells enriched from a biological sample are cultured in a closed system.

In some embodiments, the cells enriched from a biological sample are cultured in an automated system. In some embodiments, the cells enriched from a biological sample are cultured in a closed and automated system. In some embodiments, the cells enriched from a biological sample are cultured in a Terumo BCT Quantum® Cell Expansion System.

In some embodiments, the expansion of the therapeutic population of Tregs begins within 25-35 min, within 20-40 min, within 15-45 min or within 10-50 min of the enrichment from a biological sample. In some embodiments, the expansion of the therapeutic population of Tregs begins within about 30 min of the enrichment from a biological sample.

Tregs may be expanded ex vivo by culturing the cells in the presence of one or more expansion agents. In some embodiments, the expansion agent is IL-2. The appropriate concentration of IL-2 in the culture media can be determined by a person of skill in the art. In some embodiments, the concentration of IL-2 in the cell culture media is about 5-10 IU/mL, about 10-20 IU/mL, about 20-30 IU/mL, about 30-40 IU/mL, about 40-50 IU/mL, about 50-100 IU/mL, about 100-200 IU/mL, about 200-300 IU/mL, about 300-400 IU/mL, about 400-500 IU/mL, about 500-600 IU/mL, about 600-700 IU/mL, about 700-800 IU/mL, about 800-900 IU/mL, about 900-1000 IU/mL, about 1000-1500 IU/mL, about 1500-2000 IU/mL, about 2000-2500 IU/mL, about 2500-3000 IU/mL, about 3000-3500 IU/mL, about 3500-4000 IU/mL, about 4000-4500 IU/mL, about 4500-5000 IU/mL, about 5000-6000 IU/mL, about 6000-7000 IU/mL, about 7000-8000 IU/mL, about 8000-9000 IU/mL, or about 9000-10,000 IU/mL. In specific embodiments, the concentration of IL-2 in the cell culture media is about 100 IU/mL. In specific embodiments, the concentration of IL-2 in the cell culture media is about 150 IU/mL. In specific embodiments, the concentration of IL-2 in the cell culture media is about 200 IU/mL. In specific embodiments, the concentration of IL-2 in the cell culture media is about 250 IU/mL. In specific embodiments, the concentration of IL-2 in the cell culture media is about 300 IU/mL. In specific embodiments, the concentration of IL-2 in the cell culture media is about 400 IU/mL. In specific embodiments, the concentration of IL-2 in the cell culture media is about 500 IU/mL. In specific embodiments, the concentration of IL-2 in the cell culture media is about 600 IU/mL. In specific embodiments, the concentration of IL-2 in the cell culture media is about 700 IU/mL. In specific embodiments, the concentration of IL-2 in the cell culture media is about 800 IU/mL. In certain embodiments, the expansion step comprises adjusting IL-2 concentration depending on cell number. The cell number means the number of all cells in culture, including the enriched Treg cells, which represent a majority of the cells in culture and in specific embodiments represent more than 70%, more than 80%, more than 90%, more than 95%, more than 99%, or 100% of the cells in culture. In a specific embodiment, the expansion step comprises culturing the Tregs in a culture medium containing about 200 IU/mL IL-2 until the cell number reaches 600×10⁶, and then culturing the Tregs in a culture medium containing about 250 IU/mL IL-2.

In some embodiments, IL-2 is first added to the culture within about 4-5 days of initiating culture. In some embodiments, IL-2 is first added to the culture within about 3-4 days of initiating culture. In some embodiments, IL-2 is first added to the culture within about 2-3 days of initiating culture. In some embodiments, IL-2 is first added to the culture within about 1-2 days of initiating culture. In some embodiments, IL-2 is first added to the culture within about 24 hours of initiating culture. In some embodiments, IL-2 is first added to the culture within about 12 hours of initiating culture. In some embodiments, IL-2 is first added to the culture within about 6 hours of initiating culture. In some embodiments, IL-2 is first added to the culture within about 3 hours of initiating culture. In some embodiments, IL-2 is first added to the culture within about 2 hours of initiating culture. In some embodiments, IL-2 is first added to the culture within about 1 hour of initiating culture. In some embodiments, IL-2 is first added to the culture within about 30 minutes of initiating culture. In some embodiments, IL-2 is first added to the culture within about 4-5 days after the completion of the enrichment step. In some embodiments, IL-2 is first added to the culture within about 3-4 days after the completion of the enrichment step. In some embodiments, IL-2 is first added to the culture within about 2-3 days after the completion of the enrichment step. In some embodiments, IL-2 is first added to the culture within about 1-2 days after the completion of the enrichment step. In some embodiments, IL-2 is first added to the culture within about 24 hours after the completion of the enrichment step. In some embodiments, IL-2 is first added to the culture within about 12 hours after the completion of the enrichment step. In some embodiments, IL-2 is first added to the culture within about 6 hours after the completion of the enrichment step. In some embodiments, IL-2 is first added to the culture within about 3 hours after the completion of the enrichment step. In some embodiments, IL-2 is first added to the culture within about 2 hours after the completion of the enrichment step. In some embodiments, IL-2 is first added to the culture within about 1 hour after the completion of the enrichment step. In some embodiments, IL-2 is first added to the culture within about 30 minutes after the completion of the enrichment step. In some embodiments, IL-2 is replenished about every 1, 2, 3, 4, or 5 days. In some embodiments, IL-2 is replenished about every 1-2 days. In some embodiments, IL-2 is replenished about every 2-3 days. In some embodiments, IL-2 is replenished about every 3-4 days. In some embodiments, IL-2 is replenished about every 4-5 days.

In some embodiments, the expansion agent activates CD3, e.g., the expansion agent is an anti-CD3 antibody. In some embodiments, the expansion agent activates CD28, e.g., the expansion agent is an anti-CD28 antibody.

In some embodiments, the expansion agent is a soluble anti-CD3 antibody. In particular embodiments, the anti-CD3 antibody is OKT3. In some embodiments, the concentration of soluble anti-CD3 antibody in the culture media is about 0.1-0.2 ng/mL, about 0.2-0.3 ng/mL, about 0.3-0.4 ng/mL, about 0.4-0.5 ng/mL about 0.5-1 ng/mL, about 1-5 ng/mL, about 5-10 ng/mL, about 10-15 ng/mL, about 15-20 ng/mL, about 20-25 ng/mL, about 25-30 ng/mL, about 30-35 ng/mL, about 35-40 ng/mL, about 40-45 ng/mL, about 45-50 ng/mL, about 50-60 ng/mL, about 60-70 ng/mL, about 70-80 ng/mL, about 80-90 ng/mL, or about 90-100 ng/mL.

In some embodiments, the expansion agent is a soluble anti-CD28 antibody. Non-limiting examples of anti-CD28 antibodies include NA/LE (e.g. BD Pharmingen), IM1376 (e.g. Beckman Coulter), or 15E8 (e.g. Miltenyi Biotec). In some embodiments, the concentration of soluble anti-CD28 antibody in the culture media is about 1-2 ng/mL, about 2-3 ng/mL, about 3-4 ng/mL, about 4-5 ng/mL, about 5-10 ng/mL, about 10-15 ng/mL, about 15-20 ng/mL, about 20-25 ng/mL, about 25-30 ng/mL, about 30-35 ng/mL, about 35-40 ng/mL, about 40-45 ng/mL, about 45-50 ng/mL, about 50-60 ng/mL, about 60-70 ng/mL, about 70-80 ng/mL, about 80-90 ng/mL, about 90-100 ng/mL, about 100-200 ng/mL, about 200-300 ng/mL, about 300-400 ng/mL, about 400-500 ng/mL, 500-600 ng/mL, 600-700 ng/mL, about 700-800 ng/mL, about 800-900 ng/mL, or about 900-1000 ng/mL.

In some embodiments, both an anti-CD3 antibody and an anti-CD28 antibody are present in the cell culture media. In some embodiments, the anti-CD3 antibody and the anti-CD28 antibody are attached to a solid surface. In some embodiments, the anti-CD3 antibody and the anti-CD28 antibody are attached to beads. In some embodiments, beads (e.g., 3.5 μm particles) loaded with CD28 antibodies, anti-biotin antibodies and CD3-Biotin are present in the cell culture medium Such beads are commercially available (e.g., MACS GMP ExpAct Treg Kit, DYNABEADS® M-450 CD3/CD28 T Cell Expander). In specific embodiments, the ratio of anti-CD3 antibody to anti-CD28 antibody on the beads is about 100:1, 90:1, 80:1, 70:1, 60:1, 50:1, 40:1, 30:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:20, 1:30, 1:40, 1:50, 1:60, 1:70, 1:80, 1:90 or 1:100. In some embodiments, the population of Tregs is cultured in the presence of both TL-2 and beads loaded with CD28 antibodies, anti-biotin antibodies and CD3-Biotin. In some embodiments, the beads coated with anti-CD3 and anti-CD28 antibody are first added to the culture within about 4-5 days of initiating culture. In some embodiments, the beads coated with anti-CD3 and anti-CD28 antibody are first added to the culture within about 3-4 days of initiating culture. In some embodiments, the beads coated with anti-CD3 and anti-CD28 antibody are first added to the culture within about 2-3 days of initiating culture. In some embodiments, the beads coated with anti-CD3 and anti-CD28 antibody are first added to the culture within about 1-2 days of initiating culture. In some embodiments, the beads coated with anti-CD3 and anti-CD28 antibody are first added to the culture within about 24 hours of initiating culture. In some embodiments, the beads coated with anti-CD3 and anti-CD28 antibody are first added to the culture within about 12 hours of initiating culture. In some embodiments, the beads coated with anti-CD3 and anti-CD28 antibody are first added to the culture within about 6 hours of initiating culture. In some embodiments, the beads coated with anti-CD3 and anti-CD28 antibody are first added to the culture within about 3 hours of initiating culture. In some embodiments, the beads coated with anti-CD3 and anti-CD28 antibody are first added to the culture within about 2 hours of initiating culture. In some embodiments, the beads coated with anti-CD3 and anti-CD28 antibody are first added to the culture within about 1 hour of initiating culture. In some embodiments, the beads coated with anti-CD3 and anti-CD28 antibody are first added to the culture within about 30 minutes of initiating culture. In some embodiments, the beads coated with anti-CD3 and anti-CD28 antibody are first added to the culture within about 4-5 days after the completion of the enrichment step. In some embodiments, the beads coated with anti-CD3 and anti-CD28 antibody are first added to the culture within about 3-4 days after the completion of the enrichment step. In some embodiments, the beads coated with anti-CD3 and anti-CD28 antibody are first added to the culture within about 2-3 days after the completion of the enrichment step. In some embodiments, the beads coated with anti-CD3 and anti-CD28 antibody are first added to the culture within about 1-2 days after the completion of the enrichment step. In some embodiments, the beads coated with anti-CD3 and anti-CD28 antibody are first added to the culture within about 24 hours after the completion of the enrichment step. In some embodiments, the beads coated with anti-CD3 and anti-CD28 antibody are first added to the culture within about 12 hours after the completion of the enrichment step. In some embodiments, the beads coated with anti-CD3 and anti-CD28 antibody are first added to the culture within about 6 hours after the completion of the enrichment step. In some embodiments, the beads coated with anti-CD3 and anti-CD28 antibody are first added to the culture within about 3 hours after the completion of the enrichment step. In some embodiments, the beads coated with anti-CD3 and anti-CD28 antibody are first added to the culture within about 2 hours after the completion of the enrichment step. In some embodiments, the beads coated with anti-CD3 and anti-CD28 antibody are first added to the culture within about 1 hour after the completion of the enrichment step. In some embodiments, the beads coated with anti-CD3 and anti-CD28 antibody are first added to the culture within about 30 minutes after the completion of the enrichment step. In some embodiments, the beads coated with anti-CD3 and anti-CD28 antibody are again added to the culture medium about 14 days after the beads coated with anti-CD3 and anti-CD28 antibody were first added to the culture medium (e.g., if the cell number by then has not reached a target cell number). In some embodiments, the beads coated with anti-CD3 and anti-CD28 antibody are again added to the culture medium about 13 days after the beads coated with anti-CD3 and anti-CD28 antibody were first added to the culture medium (e.g., if the cell number by then has not reached a target cell number). In some embodiments, the beads coated with anti-CD3 and anti-CD28 antibody are again added to the culture medium about 12 days after the beads coated with anti-CD3 and anti-CD28 antibody were first added to the culture medium (e.g., if the cell number by then has not reached a target cell number). In some embodiments, the beads coated with anti-CD3 and anti-CD28 antibody are again added to the culture medium about 11 days after the beads coated with anti-CD3 and anti-CD28 antibody were first added to the culture medium (e.g., if the cell number by then has not reached a target cell number). In some embodiments, the beads coated with anti-CD3 and anti-CD28 antibody are again added to the culture medium about 10 days after the beads coated with anti-CD3 and anti-CD28 antibody were first added to the culture medium (e.g., if the cell number by then has not reached a target cell number). In some embodiments, the beads coated with anti-CD3 and anti-CD28 antibody are again added to the culture medium about 9 days after the beads coated with anti-CD3 and anti-CD28 antibody were first added to the culture medium (e.g., if the cell number by then has not reached a target cell number). In some embodiments, the beads coated with anti-CD3 and anti-CD28 antibody are again added to the culture medium about 8 days after the beads coated with anti-CD3 and anti-CD28 antibody were first added to the culture medium (e.g., if the cell number by then has not reached a target cell number). The cell number means the number of all cells in culture, including the enriched Treg cells, which represent a majority of the cells in culture and in specific embodiments represent more than 70%, more than 80%, more than 90%, more than 95%, more than 99%, or 100% of the cells in culture. In certain embodiments, the target cell number is 1×10⁸ to 1×10¹⁰ cells. In certain embodiments, the target cell number is 1×10⁹ to 5×10⁹ cells. In certain embodiments, the target cell number is 2×10⁹ to 5×10⁹ cells. In certain embodiments, the target cell number is 2×10⁹ to 2.5×10⁹ cells. In a specific embodiment, the target cell number is 1×10⁹ cells. In another specific embodiment, the target cell number is 1.5×10⁹ cells. In another specific embodiment, the target cell number is 2×10⁹ cells. In another specific embodiment, the target cell number is 2.5×10⁹ cells. In another specific embodiment, the target cell number is 3×10⁹ cells. In another specific embodiment, the target cell number is 3.5×10⁹ cells. In another specific embodiment, the target cell number is 4×10⁹ cells. In another specific embodiment, the target cell number is 4.5×10⁹ cells. In another specific embodiment, the target cell number is 5×10⁹ cells. In specific embodiments, the ratio of beads to cells in the culture is 10:1, 9:1, 8:1, 7:1, 6:1, 5:1, 4:1, 3:1, 2:1 or 1:1.

The expansion agent or agents may be added to the culture medium every 1, 2, 3, 4, or 5 days. In specific embodiments, the expansion agent is added to the culture medium every 1-2 days. In specific embodiments, the expansion agent is added to the culture medium every 2-3 days. In specific embodiments, the expansion agent is added to the culture medium every 3-4 days. In specific embodiments, the expansion agent is added to the culture medium every 4-5 days. In other specific embodiments, the expansion agent is added to the culture medium on day 6, 8, and 11, wherein day 0 is the day on which the biological sample is obtained from the subject. In some specific embodiments, the expansion agent is not added to the culture medium on day 13, wherein day 0 is the day on which the biological sample is obtained from the subject.

In some embodiments, the one or more expansion agents are first added to the culture within about 30 minutes-1 hour, within 1-2 hours, within 2-4 hours, within 4-6 hours, within 6-8 hours, within 8-10 hours, within 10-12 hours, within 12-14 hours, within 14-16 hours, within 16-18 hours, within 18-24 hours, within 24-36 hours, within 36-48 hours, within about 30 minutes, within about 1 hour, within about 2 hours, within about 3 hours, within about 6 hours, within about 12 hours, within about 24 hours, within about 2 days, within about 3 days, within about 4 days, within about 5 days, within about 6 days, or within about 7 days of initiating culture. In some embodiments, the one or more expansion agents are first added to the culture within about 4-5 days of initiating culture. In some embodiments, the one or more expansion agents are first added to the culture within about 3-4 days of initiating culture. In some embodiments, the one or more expansion agents are first added to the culture within about 2-3 days of initiating culture. In some embodiments, the one or more expansion agents are first added to the culture within about 1-2 days of initiating culture. In some embodiments, the one or more expansion agents are first added to the culture within about 24 hours of initiating culture. In some embodiments, the one or more expansion agents are first added to the culture within about 12 hours of initiating culture. In some embodiments, the one or more expansion agents are first added to the culture within about 6 hours of initiating culture. In some embodiments, the one or more expansion agents are first added to the culture within about 3 hours of initiating culture. In some embodiments, the one or more expansion agents are first added to the culture within about 2 hours of initiating culture. In some embodiments, the one or more expansion agents are first added to the culture within about 1 hour of initiating culture. In some embodiments, the one or more expansion agents are first added to the culture within about 30 minutes of initiating culture. In some embodiments, the one or more expansion agents are first added to the culture within about 30 minutes-1 hour, within 1-2 hours, within 2-4 hours, within 4-6 hours, within 6-8 hours, within 8-10 hours, within 10-12 hours, within 12-14 hours, within 14-16 hours, within 16-18 hours, within 18-24 hours, within 24-36 hours, within 36-48 hours, within about 30 minutes, within about 1 hour, within about 2 hours, within about 3 hours, within about 6 hours, within about 12 hours, within about 24 hours, within about 2 days, within about 3 days, within about 4 days, within about 5 days, within about 6 days, or within about 7 days after the completion of the enrichment step. In some embodiments, the one or more expansion agents are first added to the culture within about 4-5 days after the completion of the enrichment step. In some embodiments, the one or more expansion agents are first added to the culture within about 3-4 days after the completion of the enrichment step. In some embodiments, the one or more expansion agents are first added to the culture within about 2-3 days after the completion of the enrichment step. In some embodiments, the one or more expansion agents are first added to the culture within about 1-2 days after the completion of the enrichment step. In some embodiments, the one or more expansion agents are first added to the culture within about 24 hours after the completion of the enrichment step. In some embodiments, the one or more expansion agents are first added to the culture within about 12 hours after the completion of the enrichment step. In some embodiments, the one or more expansion agents are first added to the culture within about 6 hours after the completion of the enrichment step. In some embodiments, the one or more expansion agents are first added to the culture within about 3 hours after the completion of the enrichment step. In some embodiments, the one or more expansion agents are first added to the culture within about 2 hours after the completion of the enrichment step. In some embodiments, the one or more expansion agents are first added to the culture within about 1 hour after the completion of the enrichment step. In some embodiments, the one or more expansion agents are first added to the culture within about 30 minutes after the completion of the enrichment step. In some embodiments, the one or more expansion agents are again added to the culture medium about 14 days after the expansion agent(s) were first added to the culture medium. In some embodiments, the one or more expansion agents are again added to the culture medium about 13 days after the expansion agent(s) were first added to the culture medium. In some embodiments, the one or more expansion agents are again added to the culture medium about 12 days after the expansion agent(s) were first added to the culture medium. In some embodiments, the one or more expansion agents are again added to the culture medium about 11 days after the expansion agent(s) were first added to the culture medium. In some embodiments, the one or more expansion agents are again added to the culture medium about 10 days after the expansion agent(s) were first added to the culture medium. In some embodiments, the one or more expansion agents are again added to the culture medium about 9 days after the expansion agent(s) were first added to the culture medium. In some embodiments, the one or more expansion agents are again added to the culture medium about 8 days after the expansion agent(s) were first added to the culture medium.

If no expansion agent is added to the culture on a given day, that day is considered a “rest day.” In some embodiments, no expansion agent is administered during the day preceding the day on which the therapeutic population of Tregs is harvested. In some embodiments, no expansion agent is administered during the 2 days, 3 days, 4 days, 5 days or 6 days preceding the day on which the therapeutic population of Tregs is harvested.

In some embodiments, the therapeutic population of Tregs may be expanded ex vivo by culturing the cells in the presence of one or more agents that inhibit mammalian target of rapamycin (mTor). In some embodiments, the mTor inhibitor is rapamycin. In some embodiments, the mTor inhibitor is an analog of rapamycin (a “rapalog,” e.g., Temsirolimus, Everolimus, or Ridaforolimus). In some embodiments, the mTor inhibitor is ICSN3250, OSU-53, or AZD8055. In some embodiments, the concentration of rapamycin in the cell culture medium is about 1-20 nmol/L, about 20-30 nmol/L, about 30-40 nmol/L, about 40-50 nmol/L, about 50-60 nmol/L, about 60-70 nmol/L, about 70-80nmol/L, about 80-90 nmol/L, about 90-100 nmol/L, about 100-150 nmol/L, about 150-200 nmol/L, about 200-250 nmol/L, about 250-300 nmol/L, about 300-350 nmol/L, about 350-400 nmol/L, about 400-450nmol/L, about 450-500 nmol/L, about 500-600 nmol/L, about 600-700 nmol/L, about 700-800 nmol/L, about 800-900 nmo/L or about 900-1000 nmol/L. In some embodiments, the concentration of rapamycin in the cell culture media is about 100 nmol/L.

In some embodiments, the mTor inhibitor is first added to the culture within about 30 minutes-1 hour, within 1-2 hours, within 2-4 hours, within 4-6 hours, within 6-8 hours, within 8-10 hours, within 10-12 hours, within 12-14 hours, within 14-16 hours, within 16-18 hours, within 18-24 hours, within 24-36 hours, within 36-48 hours, within about 30 minutes, within about 1 hour, within about 2 hours, within about 3 hours, within about 6 hours, within about 12 hours, within about 1 day, within about 2 days, about 3 days, about 4 days, about 5 days, about 6 days, or about 7 days of initiating culture. In some embodiments, the mTor inhibitor is added to the culture medium about every 1, 2, 3, 4 or 5 days. In some embodiments, the mTor inhibitor is added to the culture medium about every 4-5 days. In some embodiments, the mTor inhibitor is added to the culture medium about every 3-4 days. In some embodiments, the mTor inhibitor is added to the culture medium about every 2-3 days. In some embodiments, the mTor inhibitor is added to the culture medium about every 1-2 days.

In certain embodiments, the expansion step takes place in a bioreactor comprising an extracapillary space. In some embodiments, flow rate of an extracapillary (EC) medium of the bioreactor can be maintained at about 0-1 mL/min, about 0-0.8 mL/min, about 0-0.6 mL/min, about 0-0.4 mL/min, about 0-0.2 mL/min, about 0.2-1 mL/min, about 0.2-0.8 mL/min, about 0.2-0.6 mL/min, about 0.2-0.4 mL/min, about 0.4-1 mL/min, about 0.4-0.8 mL/min, about 0.4-0.6 mL/min, about 0.6-1 mL/min, about 0.6-0.8 mL/min, or about 0.8-1 mL/min. In some embodiments, flow rate of an EC medium of the bioreactor can be maintained at about 0 mL/min, about 0.1 mL/min, about 0.2 mL/min, about 0.3 mL/min, about 0.4 mL/min, about 0.5 mL/min, about 0.6 mL/min, about 0.7 mL/min, about 0.8 mL/min, about 0.9 mL/min, or about 1 mL/min. In some embodiments, the expansion step comprises adjusting flow rate of an EC medium of the bioreactor depending on cell number. The cell number means the number of all cells in culture, including the enriched Treg cells, which represent a majority of the cells in culture and in specific embodiments represent more than 70%, more than 80%, more than 90%, more than 95%, more than 99%, or 100% of the cells in culture. In a specific embodiment, the expansion step comprises maintaining the flow rate of the EC medium at 0 until the cell number reaches 500×10⁶, then increasing the flow rate of the EC medium to about 0.2 mL/min and maintaining the flow rate of the EC medium at about 0.2 mL/min until the cell number reaches 750×10⁶, then increasing the flow rate of the EC medium to about 0.4 mL/min and maintaining the flow rate of the EC medium at about 0.4 mL/min until the cell number reaches about 1,000×10⁶, then increasing the flow rate of the EC medium to about 0.6 mL/min and maintaining the flow rate of the EC medium at about 0.6 mL/min until the cell number reaches about 1,500×10⁶, and then increasing the flow rate of the EC medium to about 0.8 mL/min and maintaining the flow rate of the EC medium at about 0.8 mL/min. In certain embodiments, the extracapillary medium comprises rapamycin.

The therapeutic population of Tregs may be expanded by culturing them for an appropriate duration of time. The time required for expansion resulting in a sufficiently expanded therapeutic population of Tregs for therapeutic application may be readily determined by a person of skill in the art, e.g., by monitoring the proportion of CD4⁺CD25+ cells using flow cytometry.

For example, in some embodiments, a sufficiently expanded therapeutic population of Tregs is a population of cells that contains more than 70% CD4⁺CD25+ cells as determined by flow cytometry. In some embodiments, a sufficiently expanded therapeutic population of Tregs is a population that contains about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ CD4⁺CD25+ cells per kg of body weight of the intended recipient of the therapeutic population of Tregs as determined by flow cytometry. In some embodiments, a sufficiently expanded therapeutic population of Tregs is a population that contains 1×10⁶ CD4+CD25+ cells (+/−10%) per kg of body weight of the intended recipient of the therapeutic population of Tregs as determined by flow cytometry. In some embodiments, a sufficiently expanded therapeutic population of Tregs is a population of cells that contains about or more than about 70% CD4+CD25+ cells and contains 1×10⁶ CD4+CD25+ cells (+/−10%) per kg of body weight of the intended recipient of the therapeutic population of Tregs as determined by flow cytometry.

In some embodiments, a sufficiently expanded therapeutic population of Tregs is a population that contains about 1×10⁸ to 1×10¹⁰ Treg cells. In some embodiments, a sufficiently expanded therapeutic population of Tregs is a population that contains about 1×10⁹ to 5×10⁹ Treg cells. In some embodiments, a sufficiently expanded therapeutic population of Tregs is a population that contains about 2×10⁹ to 5×10⁹ Treg cells. In some embodiments, a sufficiently expanded therapeutic population of Tregs is a population that contains about 2×10⁹ to 2.5×10⁹ Treg cells. In some embodiments, a sufficiently expanded therapeutic population of Tregs is a population that contains about 1×10⁹ Treg cells or more. In some embodiments, a sufficiently expanded therapeutic population of Tregs is a population that contains about 1.5×10⁹ Treg cells or more. In some embodiments, a sufficiently expanded therapeutic population of Tregs is a population that contains about 2×10⁹ Treg cells or more. In some embodiments, a sufficiently expanded therapeutic population of Tregs is a population that contains about 2.5×10⁹ Treg cells or more. In some embodiments, a sufficiently expanded therapeutic population of Tregs is a population that contains about 3×10⁹ Treg cells or more. In some embodiments, a sufficiently expanded therapeutic population of Tregs is a population that contains about 3.5×10⁹ Treg cells or more. In some embodiments, a sufficiently expanded therapeutic population of Tregs is a population that contains about 4×10⁹ Treg cells or more. In some embodiments, a sufficiently expanded therapeutic population of Tregs is a population that contains about 4.5×10⁹ Treg cells or more. In some embodiments, a sufficiently expanded therapeutic population of Tregs is a population that contains about 5×10⁹ Treg cells or more.

The intended recipient of the therapeutic population of Tregs may be the same subject as the donor of the biological sample from which the Tregs were enriched. Alternatively, the intended recipient of the therapeutic population of Tregs may be a subject different from the donor of the biological sample from which the Tregs were enriched.

The number of CD4+CD25+ cells may be determined every day, or every 2, 3, 4, or 5 days. In certain embodiments, if the culture does not contain a sufficiently expanded therapeutic population of Tregs on Day 15 (wherein day 0 is the day on which the biological sample is obtained from the subject), the cells may be re-activated with one or more expansion agents; while if the culture does contain a sufficiently expanded therapeutic population of Tregs on Day 15 (wherein day 0 is the day on which the biological sample is obtained from the subject), the cells may be harvested. In certain embodiments, if the culture does not contain a sufficiently expanded therapeutic population of Tregs on Day 14 (wherein day 0 is the day on which the biological sample is obtained from the subject), the cells may be re-activated with one or more expansion agents; while if the culture does contain a sufficiently expanded therapeutic population of Tregs on Day 14 (wherein day 0 is the day on which the biological sample is obtained from the subject), the cells may be harvested. In certain embodiments, if the culture does not contain a sufficiently expanded therapeutic population of Tregs on Day 13 (wherein day 0 is the day on which the biological sample is obtained from the subject), the cells may be re-activated with one or more expansion agents; while if the culture does contain a sufficiently expanded therapeutic population of Tregs on Day 13 (wherein day 0 is the day on which the biological sample is obtained from the subject), the cells may be harvested. In certain embodiments, if the culture does not contain a sufficiently expanded therapeutic population of Tregs on Day 12 (wherein day 0 is the day on which the biological sample is obtained from the subject), the cells may be re-activated with one or more expansion agents; while if the culture does contain a sufficiently expanded therapeutic population of Tregs on Day 12 (wherein day 0 is the day on which the biological sample is obtained from the subject), the cells may be harvested. In certain embodiments, if the culture does not contain a sufficiently expanded therapeutic population of Tregs on Day 11 (wherein day 0 is the day on which the biological sample is obtained from the subject), the cells may be re-activated with one or more expansion agents; while if the culture does contain a sufficiently expanded therapeutic population of Tregs on Day 11 (wherein day 0 is the day on which the biological sample is obtained from the subject), the cells may be harvested. In certain embodiments, if the culture does not contain a sufficiently expanded therapeutic population of Tregs on Day 10 (wherein day 0 is the day on which the biological sample is obtained from the subject), the cells may be re-activated with one or more expansion agents; while if the culture does contain a sufficiently expanded therapeutic population of Tregs on Day 10 (wherein day 0 is the day on which the biological sample is obtained from the subject), the cells may be harvested. In certain embodiments, if the culture does not contain a sufficiently expanded therapeutic population of Tregs on Day 9 (wherein day 0 is the day on which the biological sample is obtained from the subject), the cells may be re-activated with one or more expansion agents; while if the culture does contain a sufficiently expanded therapeutic population of Tregs on Day 9 (wherein day 0 is the day on which the biological sample is obtained from the subject), the cells may be harvested. In certain embodiments, if the culture does not contain a sufficiently expanded therapeutic population of Tregs on Day 8 (wherein day 0 is the day on which the biological sample is obtained from the subject), the cells may be re-activated with one or more expansion agents; while if the culture does contain a sufficiently expanded therapeutic population of Tregs on Day 8 (wherein day 0 is the day on which the biological sample is obtained from the subject), the cells may be harvested.

In some embodiments, a therapeutic population of Tregs is expanded by culturing for about 6-30 days, about 10-30 days, about 15-25 days, or about 18-22 days. In some embodiments, a therapeutic population of Tregs is expanded by culturing for about 15, 16, 18, 18, 19, 20, 21, 22, 23, 24, or 25 days. In certain embodiments, for example, embodiments comprising automation, partial automation or at least one automated step, a therapeutic population of Tregs is expanded by culturing for about 6-15 days, about 8-15, about 8-12 days, or about 6, 7, 8, 9, 10, 11, 12, 13, 14 or 15 days.

Viability of the cells being expanded in culture may be determined using any method known in the art. For example, the viability of cells being expanded in culture may be determined using trypan blue exclusion. Trypan blue is a dye which is excluded by cells with an intact membrane (viable cells) but taken up by cells with compromised membrane integrity (non-viable cells). Thus, viable cells appear clear under a light microscope, whereas non-viable cells appear blue. Equal amounts of trypan blue and cell suspension are mixed and counted. Viability is expressed as a percentage of trypan blue excluding cells. In some embodiments, a therapeutic population of Tregs comprises about 60%, 65% or 70% viable cells as determined by trypan blue exclusion. In some embodiments, a therapeutic population of Tregs comprises more than about 70% viable cells as determined by trypan blue exclusion. For example, in certain embodiments a therapeutic population of Tregs comprises about 75%, 80%, 85%, 90%, 95% or greater that 95% viable cells as determined by trypan blue exclusion. In some embodiments, viability of the cells being expanded in culture is determined every 2-3 days. In some embodiments, viability of the cells being expanded in culture is determined every day or every 2, 3, 4, or 5 days.

In some embodiments, the cells are washed one or more times during the culturing to remove agents present during the incubation or culturing and/or to replenish the culture medium with one or more additional agents. In some embodiments, the cells are washed during the incubation or culturing to reduce or remove the expansion agent(s). The culture medium may be replaced about every 2, 3, 4, 5, 6 or 7 days, for example, every 2-3 days or every 3-4 days. In some embodiments, only part of the culture medium (e.g., about 50% of the culture medium) is replaced. In other embodiments, the entire culture medium is replaced. In some embodiments, the cell culture is not centrifuged during a change of culture medium. In some embodiments, the cell culture is not centrifuged during harvesting.

The therapeutic population of Tregs may be harvested by any means known in the art, for example, by centrifugation. In some embodiments, the therapeutic population of Tregs is harvested on day 8, wherein day 0 is the day on which the biological sample is obtained from the subject. In some embodiments, the therapeutic population of Tregs is harvested on day 9, wherein day 0 is the day on which the biological sample is obtained from the subject. In some embodiments, the therapeutic population of Tregs is harvested on day 10, wherein day 0 is the day on which the biological sample is obtained from the subject. In some embodiments, the therapeutic population of Tregs is harvested on day 11, wherein day 0 is the day on which the biological sample is obtained from the subject. In some embodiments, the therapeutic population of Tregs is harvested on day 12, wherein day 0 is the day on which the biological sample is obtained from the subject. In some embodiments, the therapeutic population of Tregs is harvested on day 13, wherein day 0 is the day on which the biological sample is obtained from the subject. In some embodiments, the therapeutic population of Tregs is harvested on day 14, wherein day 0 is the day on which the biological sample is obtained from the subject. In some embodiments, the therapeutic population of Tregs is harvested on day 15, wherein day 0 is the day on which the biological sample is obtained from the subject. In some embodiments, the therapeutic population of Tregs is harvested on day 19, wherein day 0 is the day on which the biological sample is obtained from the subject In some embodiments, the therapeutic population of Tregs is harvested on day 20, wherein day 0 is the day on which the biological sample is obtained from the subject. In some embodiments, the therapeutic population of Tregs is harvested on day 25, wherein day 0 is the day on which the biological sample is obtained from the subject. In some embodiments, the therapeutic population of Tregs is harvested on day 16, 17 or 18, wherein day 0 is the day on which the biological sample is obtained from the subject. In some embodiments, the therapeutic population of Tregs is harvested on day 21, 22, 23, or 24, wherein day 0 is the day on which the biological sample is obtained from the subject.

In some aspects the population of Tregs is subjected to genetic engineering at any point of the method prior to cryopreservation. In some embodiments, the population of Tregs is subjected to genetic engineering two or more times at any point prior to cryopreservation. In some embodiments, the engineering may comprise the introduction of a transgene into the Tregs or the introduction of an mRNA into the Tregs. In some embodiments, the genetic engineering may also comprise the gene editing through CRISPR-Cas9. In some embodiments, the genetic engineering comprises the reduction of gene expression through siRNA or antisense oligonucleotides. In some embodiments, the genetic engineering may allow for the use of a therapeutic population of Tregs provided herein in an allogeneic setting. In some embodiments, the genetic engineering introduces a chimeric antigen receptor (CAR) into the Tregs.

### 7.2 Methods of Cryopreserving and Thawing Tregs

In another aspect, provided herein is a cryopreserved population of Tregs having characteristics as described herein. A cryopreserved therapeutic population of Tregs may be produced by the methods described herein. Also disclosed herein are pharmaceutical compositions comprising a cryopreserved therapeutic population of Tregs that has been thawed and is present in a formulation suitable for administration to a subject, for example a human subject. In certain embodiments, the formulation comprises a pharmaceutically acceptable carrier, e.g., normal saline. In certain embodiments, the formulation comprises human serum albumin. In other embodiments, the formulation comprises normal saline and human serum albumin.

In some embodiments, a therapeutic population of Tregs is cryopreserved after expansion. The therapeutic population of Tregs may be cryopreserved in any suitable medium known in the art. Examples of media suitable for cryopreservation include, e.g., CryoStor® CS10. In some embodiments, a therapeutic population of Tregs is frozen in a composition comprising a cryoprotectant, for example, in a composition comprising DMSO (e.g., 10% DMSO). In some embodiments, the therapeutic population of Tregs is frozen in a comprising glycerol. In some embodiments, the cryoprotectant is or comprises DMSO and/or glycerol.

In some embodiments, a therapeutic population of Tregs may be stored at about −200° C. to −190° C., about −180 to −140° C., or about −90 to −70° C. In some embodiments, a therapeutic population of Tregs may be stored at about −196° C. In some embodiments, a therapeutic population of Tregs may be stored at about −80° C. In some embodiments, a therapeutic population of Tregs may be stored in the liquid nitrogen vapor phase. In some embodiments, a therapeutic population of Tregs may be stored on frozen carbon dioxide (dry ice).

In another aspect, a cryopreserved therapeutic population of Tregs may be stored at a first temperature for a period of time, for example an extended period of time, e.g., about month, about 3 months, about 6 months, about 9 months, about 12 months, about 18 months, or about 24 months, and subsequently stored at a second temperature for a shorter period of time (e.g., about 6 hours, about 12 hours, about 24 hours, about 36 hours, or about 48 hours). In another aspect, a cryopreserved therapeutic population of Tregs may be stored at a first temperature for a period of time, e.g., about 6 hours, about 12 hours, about 24 hours, about 36 hours, or about 48 hours, subsequently stored at a second temperature for a longer period of time e.g., about month, about 3 months, about 6 months, about 9 months, about 12 months, about 18 months, or about 24 months. In some embodiments, the first temperature is lower than the second temperature. In some embodiments, the first temperature is about −200° C. to −190° C., about −180 to −140° C., about −90 to −70° C., about −196° C. or about −80° C. In some embodiments, the second temperature is about −80° C. or about −20° C.

In some embodiments, a therapeutic population of Tregs is stored at about −196° C. for about 1 month, about 3 months, about 6 months, about 9 months, about 12 months, about 18 months, or about 24 months and subsequently stored on frozen carbon dioxide for about 6 hours, about 12 hours, about 24 hours, about 36 hours, or about 48 hours. In some embodiments, a therapeutic population of Tregs is stored in the liquid nitrogen vapor phase for about 1 month, about 3 months, about 6 months, about 9 months, about 12 months, about 18 months, or about 24 months and subsequently stored on frozen carbon dioxide for about 6 hours, about 12 hours, about 24 hours, about 36 hours, or about 48 hours.

In some embodiments, the therapeutic population of Tregs is cryopreserved at a high Treg density. In specific embodiments, the therapeutic population of Tregs is cryopreserved at a concentration of 1×10⁶ cells (+/−10%) per kg of body weight of the intended recipient of the Tregs. The intended recipient of the Tregs may be the same or different individual as the subject from whom the initial biological sample containing Tregs was obtained (the donor). In some embodiments, the cryopreserved therapeutic population of Tregs is stored at a density of at 10 million, at least 20 million, at least 25 million, at least 30 million, at least 35 million, at least 40 million, at least 45 million, at least 50 million, at least 55 million, at least 60 million, at least 65 million, at least 70 million, at least 75 million, at least 80 million, at least 85 million, at least 90 million, at least 95 million, or at least 100 million cells per mL.

In some embodiments, the therapeutic population of Tregs is cryopreserved in a cryovial. In some embodiments, the therapeutic population of Tregs is frozen in a cryovial in a volume of about 0.5 mL to 1 mL, about 1 mL to 1.5 mL, or about 1.5 mL to 2 mL, about 2-5 mL, about 5-10 mL, or about 15-20 mL. In some embodiments, the therapeutic population of Tregs is frozen in a cryovial in a volume of about 1.0 mL, about 1.1 mL, about 1.2 mL, about 1.3 mL, about 1.4 mL, about 1.5 mL, about 1.6 mL, about 1.7 mL, about 1.8 mL, about 1.8 mL, about 1.9 mL or about 2.0 mL.

In some embodiments, the therapeutic population of Tregs is frozen in a cryopreservation bag, e.g., a gas permeable bag. In some embodiments, the therapeutic population of Tregs is frozen in a cryopreservation bag in a volume of about 1-2 mL, about 2-5 mL, about 5-10 mL, about 10-15 mL, or about 15-20 mL. In some embodiments, the therapeutic population of Tregs is frozen in a cryopreservation bag in a volume of about 1 mL, about 2 mL, about 5 mL, about 10 mL or about 20 mL.

In some embodiments, cryopreservation comprises decreasing the temperature of the therapeutic population of Tregs in the following increments: 1° C./min to 4° C., 25° C./min to −40° C., 10° C./min to −12° C., 1° C./min to −40° C., and 10° C./min to −80° C. to −90° C.

The cryopreserved therapeutic population of Tregs may be thawed, for example, about 1 day, about 2 days, about 3 days, about 4 days, about 5 days, about 6 days, about 1 week, about 1-2 weeks, about 2-4 weeks, about 1 month, about 1-2 months, about 2-3 months, about 3-6 months, about 6-9 months, about 9-12 months, about 12-15 months, about 15-18 months, about 18-24 months, about 1-2 years, about 2-3 years, about 3-4 year or about 4-5 years after cryopreservation. In some embodiments, the cryopreserved therapeutic population of Tregs may be thawed, for example, about 1 week, 1 month, about 3 months, about 6 months, about 9 months, about 12 months or about 18 months after cryopreservation.

In some embodiments, the cryopreserved therapeutic population of Tregs is thawed using a method that results in at least 60%, at least 70%, at least 75%, at least 80%, at least 85%, at least 90%, at least 95% or greater than 95% viability of the Tregs as determined, for example, by Trypan Blue exclusion.

In some embodiments, the cryopreserved therapeutic population of Tregs is thawed and diluted in a solution comprising 0.9% sodium chloride. In some embodiments, the cryopreserved therapeutic population of Tregs is thawed and diluted in a solution comprising 0.9% sodium chloride and about 5% human serum albumin. In some embodiments, the cryopreserved therapeutic population of Tregs is thawed and diluted in a solution wherein the resulting solution comprises 0.9% sodium chloride. In some embodiments, the cryopreserved therapeutic population of Tregs is thawed and diluted in a solution wherein the resulting solution comprises 0.9% sodium chloride and about 5% human serum albumin. In certain embodiments, the cryopreserved therapeutic population of Tregs is thawed and without further expansion is placed into a solution as described herein.

In some embodiments, the cryopreserved therapeutic population of Tregs is thawed and diluted in 50 mL of a solution comprising 0.9% sodium chloride and about 5% human serum albumin. In some embodiments, the cryopreserved therapeutic population of Tregs is thawed and diluted in a solution, wherein the resulting solution is a 50 mL solution comprising 0.9% sodium chloride and about 5% human serum albumin. In certain embodiments, the cryopreserved therapeutic population of Tregs is thawed and without further expansion is placed into a solution as described herein.

In some embodiments, one cryovial containing the cryopreserved therapeutic population of Tregs is thawed and placed in 50 mL of a solution comprising 0.9% sodium chloride and about 5% human serum albumin. In some embodiments, one cryovial containing the cryopreserved therapeutic population of Tregs is thawed and placed in solution, wherein the resulting solution is a 50 mL solution comprising 0.9% sodium chloride and about 5% human serum albumin. In certain embodiments, the cryopreserved therapeutic population of Tregs is thawed and without further expansion is placed into a solution as described herein.

In some embodiments, the cryopreserved therapeutic population of Tregs is thawed using an automated thawing system (e.g., a COOK regentec thawing system). In some embodiments, the cryopreserved therapeutic populations of Tregs is rapidly thawed. In some embodiments, the cryopreserved therapeutic population of Tregs is thawed at a controlled rate. In some embodiments, the cryopreserved therapeutic population of Tregs is thawed in a water bath. In some embodiments, the cryopreserved therapeutic population of Tregs is thawed at room temperature.

In some embodiments, the therapeutic population of Tregs is administered to a subject within about 2-10 hours, within about 4-8 hours, or within about 5-7 hours of thawing. In some embodiments, the therapeutic population of Tregs is administered to a subject within about 30 minutes, or about 1, 2, 3, 4, 5, 6, 7, 8, 9, 10, 11 or 12 hours of thawing. In specific embodiments, the therapeutic population of Tregs is administered to the subject within about 6 hours of thawing.

In some embodiments, the cryopreserved therapeutic population of Tregs is thawed and administered to the patient without further dilution. In some embodiments, the cryopreserved therapeutic population of Tregs is thawed and administered to the patient without further dilution or further expansion. In some embodiments, the cryopreserved therapeutic population of Tregs is thawed and administered to the patient without further dilution and in combination with normal saline. In some embodiments, the cryopreserved therapeutic population of Tregs is thawed and administered to the patient without further dilution or further expansion and in combination with normal saline. In some embodiments, the thawed cryopreserved therapeutic population of Tregs and normal saline are administered concurrently and intravenously.

In some embodiments, no further expansion of the therapeutic population of Tregs is required between thawing and administration to the subject. In some embodiments, no further expansion of the therapeutic population of Tregs is performed between thawing and administration to the subject.

### 7.2.1. Automation

In some embodiments, a method of producing a therapeutic population of Tregs can be carried out in a closed system. The methods in some embodiments are carried out in an automated or partially automated fashion. For example, the method of producing a therapeutic population of Tregs described herein (e.g., the method described in Section 7.1 or 8.1 herein) may be carried out in a bioreactor. In some embodiments, the method is carried out in a G-REX®culture system. In some embodiments, the method is carried out in a Terumo BCT Quantum® Cell Expansion System. FIG. 1 shows an exemplary process of producing a therapeutic population of Tregs in a bioreactor.

In some embodiments, any one or more of the steps of the method of producing a therapeutic population of Tregs can be carried out in a closed system or under GMP conditions. In some embodiments, one or more or all of the steps (e.g., enrichment and/or expansion) is carried out using a system, device, or apparatus in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the steps

In certain embodiments, the enrichment step is automated. In certain embodiments, the enrichment step takes place in a closed system. In certain embodiments, the enrichment step is automated and takes place in a closed system. In specific embodiments, the enrichment step takes place in a CliniMACS Prodigy® system. In specific embodiments, the enrichment step takes place in a CliniMACS® Plus system. In certain embodiments, the expansion step is automated. In certain embodiments, the expansion step takes place in a closed system. In certain embodiments, the expansion step is automated and takes place in a closed system. In specific embodiments, the expansion step takes place in a bioreactor (e.g., a Terumo BCT Quantum® Cell Expansion System). In some embodiments, the enrichment step and the expansion step take place in different systems (for example, the enrichment step takes place in a CliniMACS Prodigy® system and the expansion step takes place in a Terumo BCT Quantum® Cell Expansion System, or the enrichment step takes place in a CliniMACS® Plus system and the expansion step takes place in a Terumo BCT Quantum® Cell Expansion System). In specific embodiments, the enriched cell population produced by the enrichment step are transferred to the system where the expansion step takes place in a closed step. In other embodiments, the enrichment step and the expansion step take place in the same system. In specific embodiments, the same system is a closed system.

In some embodiments, the method of producing a therapeutic population of Tregs comprises a step of expanding the Tregs. In some embodiments, the expansion step is automated. In some embodiments, the Tregs are expanded for 6, 7, 8, 9, 10, 11, or 12 days. In one embodiment, the Tregs are expanded for 8 days. In another embodiment, the Tregs are expanded for 11 days. In another embodiment, the Tregs are expanded for 13, 14, or 15 days. In another embodiment, the Tregs are expanded for 15 days.

In some embodiments, the method of producing a therapeutic population of Tregs comprises administering an expansion agent (such as IL-2, a CD3-activating agent and/or a CD28-activating agent) every 1, 2, 3, 4, 5, 6, 7, 8, 9 or 10 days. In some embodiments, the expansion agent (such as IL-2, a CD3-activating agent and/or a CD28-activating agent) is first administered within 24 h of initiating the culture. In some embodiments, the expansion agent (such as IL-2, a CD3-activating agent and/or a CD28-activating agent) is first administered within 12 h of initiating the culture. In some embodiments, the expansion agent (such as IL-2, a CD3-activating agent and/or a CD28-activating agent) is first administered within 6 h of initiating the culture. In some embodiments, the expansion agent (such as IL-2, a CD3-activating agent and/or a CD28-activating agent) is first administered within 3 h of initiating the culture. In some embodiments, the expansion agent (such as IL-2, a CD3-activating agent and/or a CD28-activating agent) is first administered within 2 h of initiating the culture. In some embodiments, the expansion agent (such as IL-2, a CD3-activating agent and/or a CD28-activating agent) is first administered within 1 h of initiating the culture. In some embodiments, the expansion agent (such as IL-2, a CD3-activating agent and/or a CD28-activating agent) is first administered within 30 minutes of initiating the culture. In some embodiments, the expansion agent (such as IL-2, a CD3-activating agent and/or a CD28-activating agent) is administered daily. In some embodiments, the method of producing a therapeutic population of Tregs comprises replacing the culture medium every 1, 2, 3, 4, 5, 6 or 7 days. In some embodiments, the media may be changed when the level of certain metabolites (e.g., lactacte) reach a predetermined threshold. In some embodiments, the media changes based on the expansion rate of the therapeutic population of Tregs.

In some embodiments, the concentration of cells in the culture is determined on Day 8. In some embodiments, the concentration of cells in the culture is determined on Day 11. In some embodiments, the concentration of cells in the culture is determined on Day 15. In some embodiments, the system remains closed throughout the expansion step.

### 7.3 Compositions

Provided herein are compositions comprising a therapeutic population of Tregs suitable for administration to a subject. In certain embodiments, the methods of producing a cryopreserved therapeutic population of Tregs provided herein further comprise thawing the cryopreserved therapeutic population of Tregs and, without further expansion, placing the population into a composition comprising a pharmaceutically acceptable carrier, to produce a pharmaceutical composition. In some embodiments, provided herein is a pharmaceutical composition produced by a method described herein. In some embodiments, provided herein is a cryopreserved therapeutic population of Tregs produced by a method described herein. In some embodiments, provided herein is a pharmaceutical composition comprising a thawed and unexpanded form of a cryopreserved therapeutic population of Tregs described herein, and a pharmaceutically acceptable carrier. In some embodiments, provided herein is a cryopreserved composition comprising a therapeutic population of Tregs having characteristics as described herein. A therapeutic population of Tregs, for example, a cryopreserved therapeutic population of Tregs, may be produced by the methods described herein. Also disclosed herein are pharmaceutical compositions comprising a cryopreserved therapeutic population of Tregs that has been thawed and is present in a formulation suitable for administration to a subject, for example a human subject.

In some embodiments, a therapeutic population of Tregs provided herein comprises about 60%, 65%, 70%, 75%, 80%, 85%, 90%, 95% or greater than 95% viable cells as determined by trypan blue exclusion. In some embodiments, a therapeutic population of Tregs provided herein comprises more than about 70% viable cells.

In some embodiments, a therapeutic population of Tregs provided herein comprises about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10′ to about 8×10⁸, about 8×10⁸ to about 9×10⁸, about 9×10⁸ to about 1×10⁹ CD4⁺CD25+ cells.

In some embodiments, a therapeutic population of Tregs provided herein comprises about 1×10⁸ to 1×10¹⁰ Treg cells. In some embodiments, a therapeutic population of Tregs provided herein comprises about 1×10⁹ to 5×10⁹ Treg cells. In some embodiments, a therapeutic population of Tregs provided herein comprises about 2×10⁹ to 5×10⁹ Treg cells. In some embodiments, a therapeutic population of Tregs provided herein comprises about 2×10⁹ to 2.5×10⁹ Treg cells. In some embodiments, a therapeutic population of Tregs provided herein comprises about 1×10⁹ Treg cells or more. In some embodiments, a therapeutic population of Tregs provided herein comprises about 1.5×10⁹ Treg cells or more. In some embodiments, a therapeutic population of Tregs provided herein comprises about 2×10⁹ Treg cells or more. In some embodiments, a therapeutic population of Tregs provided herein comprises about 2.5×10⁹ Treg cells or more. In some embodiments, a therapeutic population of Tregs provided herein comprises about 3×10⁹ Treg cells or more. In some embodiments, a therapeutic population of Tregs provided herein comprises about 3.5×10⁹ Treg cells or more. In some embodiments, a therapeutic population of Tregs provided herein comprises about 4×10⁹ Treg cells or more. In some embodiments, a therapeutic population of Tregs provided herein comprises about 4.5×10⁹ Treg cells or more. In some embodiments, a therapeutic population of Tregs provided herein comprises about 5×10⁹ Treg cells or more.

In some embodiments, a therapeutic population of Tregs provided herein comprises about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ CD4⁺CD25+ cells per mL.

In some embodiments, a therapeutic population of Tregs provided herein comprises about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ CD4⁺CD25+ cells per kg of body weight of an intended recipient subject as determined by flow cytometry. In some embodiments, a therapeutic population of Tregs provided herein comprises 1×10⁶ CD4⁺CD25+ cells (+/−10%) per kg of body weight of the subject as determined by flow cytometry. In some embodiments, a therapeutic population of Tregs provided herein comprises about or more than about 70% CD4⁺CD25+ cells and contains 1×10⁶ CD4⁺CD25+ cells (+/−10%) per kg of body weight of the subject as determined by flow cytometry. The subject may be the same subject as the donor of the biological sample from which the Tregs were enriched. Alternatively, the subject may be a subject different from the donor of the biological sample from which the Tregs were enriched.

A cryopreserved therapeutic population of Tregs provided herein may comprise an increased proportion of CD4⁺CD25^{high} Tregs relative to the proportion of CD4⁺CD25^{high} Tregs in a corresponding baseline Treg cell population, as determined by flow cytometry. In some embodiments, a cryopreserved therapeutic population of Tregs comprises an increased proportion of CD4⁺CD25^{high}CD127^{low} Tregs relative to the proportion of CD4⁺CD25^{high}CD127^{low} Tregs in the baseline Treg cell population, as determined by flow cytometry. In some embodiments, an therapeutic population of Tregs provided herein may comprise an increased proportion of CD4⁺CD25^{high} Tregs relative to the proportion of CD4⁺CD25^{high} Tregs in the baseline Treg cell population, as determined by flow cytometry. In some embodiments, an expanded therapeutic population of Tregs comprises an increased proportion of CD4⁺CD25^{high}CD127^{low} Tregs relative to the proportion of CD4⁺CD25^{high}CD127^{low} Tregs in the baseline Treg cell population, as determined by flow cytometry. In this context, “high” and “low” denote the expression level of a marker (e.g., CD25 or CD127) in a subpopulation of cells relative to the entire population.

In some embodiments, the cryopreserved therapeutic population of Tregs comprises CD25+ Tregs wherein the expression of CD25 in the Tregs is increased relative to the expression of CD25 in the Tregs in the baseline Treg cell population, as determined by flow cytometry. The expression of CD25 is increased by at least about 5-fold, at least about 10-fold, at least about 15-fold, at least about 20-fold, at least about 25-fold, at least about 30-fold, at least about 35-fold, at least about 40-fold, at least about 45-fold or at least about 50-fold as determined by flow cytometry.

In some embodiments, the cryopreserved population of Tregs comprises CD127+Tregs wherein the expression of CD127 in the Tregs is not increased greater than 3-fold relative to the expression of CD127 in the Tregs in the baseline Treg cell population, as determined by flow cytometry. In some embodiments, the expression of CD127 is not increased relative to the expression of CD127 in the Tregs in the Treg-enriched cell population, as determined by flow cytometry.

In some embodiments, the cryopreserved therapeutic population of Tregs comprises at least 70%, at least 80%, or at least 90% CD4⁺CD25^{high}CD127° Tregs in the baseline Treg cell population, as determined by flow cytometry. In some embodiments, the cryopreserved therapeutic population of Tregs comprises at least 70%, at least 75%, at least 80%, at least 85%, at least 90% or at least 95% CD4⁺CD25+ cells, as determined by flow cytometry. In some embodiments, a composition comprising a therapeutic population of Tregs provided herein comprises less than 20% CD8+ cells and comprises 1×10⁶ CD4+CD25+ cells (+/−10%) per kg of body weight of the subject as determined by flow cytometry. In some embodiments, a composition comprising a therapeutic population of Tregs provided herein comprises less than 20% CD8+ cells, comprises about or more than about 70% CD4⁺CD25+ cells, and comprises 1×10⁶ CD4+CD25+ cells (+/−10%) per kg of body weight of the subject as determined by flow cytometry.

In some embodiments, the granularity of the Tregs in the cryopreserved therapeutic population of Tregs is increased relative to the granularity of the Tregs in the Treg-enriched cell population, as determined by flow cytometry. In some embodiments, the granularity of the Tregs is increased by at least about 1.5-fold, at least about 2-fold, or at least about 2.5-fold.

In some embodiments, the size of the Tregs in the cryopreserved therapeutic population of Tregs is increased relative to the size of the Tregs in the baseline Treg cell population, as determined by flow cytometry. In some embodiments, the size of the Tregs is increased by at least about 1.2-fold, at least about 1.5-fold, or at least about 2-fold.

In some embodiments, the cryopreserved therapeutic population of Tregs comprises CTLA4+ Tregs wherein the proportion of CTLA4+ Tregs is increased relative to the proportion of CTLA4+ Tregs in the baseline Treg cell population. In some embodiments, an expanded therapeutic population of Tregs comprises CTLA4+ Tregs wherein the proportion of CTLA4+Tregs is increased relative to the proportion of CTLA4+ Tregs in the baseline Treg cell population. In some embodiments, the cryopreserved therapeutic population of Tregs comprises at least 10%, at least 20%, at least 30%, at least 40%, at least 50%, at least 60%, at least 70%, at least 80%, or at least 90% CTLA4+ Tregs, as determined by flow cytometry.

In some embodiments, the cryopreserved therapeutic population of Tregs comprises FoxP3+ Tregs wherein the proportion of FoxP3+ Tregs is increased relative to the proportion of FoxP3+ Tregs in the baseline Treg cell population. In some embodiments, an expanded therapeutic population of Tregs comprises FoxP3+ Tregs wherein the proportion of FoxP3+ Tregs is increased relative to the proportion of FoxP3+ Tregs in the baseline Treg cell population. In some embodiments, the cryopreserved therapeutic population of Tregs comprises at least 10%, at last 20%, at last 30%, at least 40%, at least 50%, at least 60%, at least 70%, or at least 80% or at least 90% FoxP3+ Tregs, as determined by flow cytometry.

In some embodiments, the Tregs in the cryopreserved therapeutic population of Tregs comprise FoxP3-expressing Tregs wherein the expression of FoxP3 is increased in the Tregs relative to expression of FoxP3 in the Tregs in the baseline Treg cell population prior to expansion. In some embodiments, a therapeutic population of Tregs or a cryopreserved composition comprising a therapeutic population of Tregs provided herein expresses high levels of FoxP3, wherein the one or more regulatory element (e.g., a promoter or an enhancer) of the FOXP3 gene is demethylated. In some embodiments, the Treg specific demethylated region (TSDR) within FOXP3 is demethylated. In some embodiments, the expression of one or more gene products associated with FOXP3 demethylation is increased in the therapeutic population of Tregs or in the cryopreserved therapeutic population of Tregs after expansion. In some embodiments, one or more gene products associated with FOXP3 methylation is decreased in the therapeutic population of Tregs or in the cryopreserved therapeutic population of Tregs after expansion.

In some embodiments, a cryopreserved population of Tregs expresses high levels of glucocorticoid-induced tumor necrosis factor receptor (GITR).

In some embodiments, a cryopreserved population of Tregs comprises less than 20% CD8+ cells, determined by flow cytometry.

In some embodiments, the viability of the cryopreserved therapeutic population of Tregs is at least 70%, at least 75%, at least 80%, at least 85%, at least 90% or at least 95%, as determined by trypan blue staining performed following thawing of the cryopreserved therapeutic population. In some embodiments, the viability of the cryopreserved therapeutic population of Tregs, as determined by trypan blue staining performed following thawing of the cryopreserved therapeutic population, is at least 70%, at least 75%, at least 80%, at least 85%, at least 90% or at least 95% of the viability of the expanded Treg cell population prior to the expanded Treg cell population being cryopreserved. In some embodiments, the viability of the therapeutic population of Tregs, as determined by trypan blue staining performed before cryopreserving the therapeutic population of Tregs is increased compared to the viability of the enriched Treg cell population prior to expansion.

In some embodiments, the suppressive function of the cryopreserved population of Tregs is at least 60%, at least 70%, at least 75%, at least 80%, at least 85%, at least 90% or at least 95%, as determined by suppression of proliferation of responder T cells by flow cytometry or thymidine incorporation performed following thawing of the therapeutic population. Suppressive function of Tregs may be assessed, for example by measuring the proliferation of CFSE positive responder T cells by flow cytometry or thymidine incorporation. CFSE is an intracellular marker only present in the responder T cell population. Responder T cells are generally characterized as CD4⁺CD25-T cells and may be isolated using the CD4⁺CD25+Regulatory T Cell Isolation Kit (Miltenyi Biotec). For example, a sample maybe separated into a positively selected cell fraction containing CD4⁺CD25+regulatory T cells and the unlabeled CD4⁺CD25-cell effluent which contains the responder T cell population.

In some embodiments, the cryopreserved population of Tregs exhibits a suppressive function that is greater than the suppressive function of the enriched Treg cell population, as measured prior to expansion, as suppression of proliferation of responder T cells by flow cytometry or thymidine incorporation. In some embodiments, the suppressive function of the cryopreserved therapeutic population of Tregs, as determined by suppression of proliferation of responder T cells by flow cytometry or thymidine incorporation performed following thawing of the therapeutic population, is at least 60%, at least 70%, at least 75%, at least 80%, at least 85%, at least 90% or at least 95% of the suppressive function of the expanded Treg cell population prior to the Tregs being cryopreserved, as determined by flow cytometry. In some embodiments, the suppressive function of the therapeutic population of Tregs, as determined by suppression of proliferation of responder T cells by flow cytometry or thymidine incorporation performed before cryopreserving the therapeutic population of Tregs is increased compared to the suppressive function of the enriched Treg cell population prior to expansion.

In some embodiments, the cryopreserved therapeutic population of Tregs exhibits an ability to suppress inflammatory cells, as measured by IL-6 production by the inflammatory cells (e.g., macrophages or monocytes from human donors or generated from induced pluripotent stem cells). In some embodiments, the cryopreserved therapeutic population of Tregs exhibits an ability to suppress the function of myeloid cells (e.g., macrophages, monocytes, or microglia). In some embodiments, the cryopreserved therapeutic population of Tregs exhibits an ability to suppress the release of inflammatory cytokines (e.g., IL-1β, IL-6, IL-8, TNFα, or INFγ).

In some embodiments, the expanded therapeutic population of Tregs exhibits an ability to suppress inflammatory cells, as measured by IL-6 production by the inflammatory cells (e.g., macrophages or monocytes from human donors or generated from induced pluripotent stem cells). In some embodiments, the expanded therapeutic population of Tregs exhibits an ability to suppress the function of myeloid cells (e.g., macrophages, monocytes, or microglia). In some embodiments, the expanded therapeutic population of Tregs exhibits an ability to suppress the release of inflammatory cytokines (e.g., IL-1β, IL-6, IL-8, TNFα, or INFγ).

In specific embodiments, the cryopreserved population of Tregs exhibits an improved ability to suppress secretion of inflammatory cytokines (e.g., IL-1β, IL-6, IL-8, TNFα, or INFγ) from macrophages relative to the therapeutic population of Tregs prior to expansion. In other specific embodiments, the cryopreserved population of Tregs exhibits an improved ability to suppress secretion of inflammatory cytokines (e.g., IL-1β, IL-6, IL-8, TNFα, or INFγ) from monocytes relative to the therapeutic population of Tregs prior to expansion. In other specific embodiments, the cryopreserved population of Tregs exhibits an improved ability to suppress secretion of inflammatory cytokines (e.g., IL-1β, IL-6, IL-8, TNFα, or INFγ) from microglia relative to the therapeutic population of Tregs prior to expansion.

In specific embodiments, the expanded population of Tregs prior to cryopreservation exhibits an improved ability to suppress secretion of inflammatory cytokines (e.g., IL-1β, IL-6, IL-8, TNFα, or INFγ) from macrophages relative to the therapeutic population of Tregs prior to expansion. In other specific embodiments, the expanded population of Tregs prior to cryopreservation exhibits an improved ability to suppress secretion of inflammatory cytokines (e.g., IL-1β, IL-6, IL-8, TNFα, or INFγ) from monocytes relative to the therapeutic population of Tregs prior to expansion. In other specific embodiments, the expanded population of Tregs prior to cryopreservation exhibits an improved ability to suppress secretion of inflammatory cytokines (e.g., IL-1β, IL-6, IL-8, TNFα, or INFγ) from microglia relative to the therapeutic population of Tregs prior to expansion.

In some embodiments, the cryopreserved population of Tregs is thawed in accordance with a method described herein.

### 7.3.1. Pharmaceutical Compositions

In certain aspects, provided are pharmaceutical compositions comprising a therapeutic population of Tregs as described herein. In certain embodiments, provided herein is a pharmaceutical composition comprising a therapeutic population of Tregs as described herein and a buffer. In some embodiments, the pharmaceutical composition comprises a therapeutic population of Tregs provided herein in a sterile buffer.

In some embodiments, a pharmaceutical composition provided herein comprises a therapeutic population of Tregs in a buffer suitable for administration to a human subject. Examples of buffers suitable for administration to a human subject include saline-containing buffers such as phosphate buffered saline, physiological saline, normal saline or 0.9% saline. Thus, in certain embodiments, a pharmaceutical composition provided herein comprises a therapeutic population of Tregs in a sterile buffer, e.g., a saline-containing buffer. In particular embodiments, the pharmaceutical composition comprises a therapeutic population of Tregs and physiological saline. In particular embodiments, the pharmaceutical composition comprises a therapeutic population of Tregs and normal saline. In particular embodiments, the pharmaceutical composition comprises a therapeutic population of Tregs and 0.9% saline. In particular embodiments, the pharmaceutical composition comprises a therapeutic population of Tregs and phosphate-buffered saline.

In some embodiments, a composition provided herein is a pharmaceutical composition comprising a therapeutic population of Tregs provided herein and a pharmaceutically acceptable carrier, excipient, or diluent. In some embodiments, provided herein is a pharmaceutical composition comprising an effective amount of a therapeutic population of Tregs provided herein and a carrier, excipient, or diluent, that is, an amount of therapeutic population of Tregs provided herein which is sufficient to result in a desired outcome.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

The carrier, excipient, or diluent may be any pharmaceutically acceptable carrier, excipient or diluent, known in the art. Examples of pharmaceutically acceptable carriers include non-toxic solids, semisolids, or liquid fillers, diluents, encapsulating materials, formulation auxiliaries or carriers. A pharmaceutically acceptable carrier can include all solvents, dispersion media, coatings, antibacterial and antifungal agents, isotonic and absorption delaying agents, and the like, compatible with pharmaceutical administration. Examples of such carriers or diluents include, but are not limited to, water, saline, Ringer's solutions, dextrose solution, and 5% human serum. Liposomes and non-aqueous vehicles such as fixed oils may also be used.

Excipients may include, for example, encapsulating materials or additives such as absorption accelerators, antioxidants, binders, buffers, coating agents, coloring agents, disintegrating agents, emulsifiers, extenders, fillers, flavoring agents, humectants, lubricants, perfumes, preservatives, propellants, releasing agents, sterilizing agents, sweeteners, solubilizers, wetting agents, and mixtures thereof. The term “excipient” may itself refer to a carrier or diluent.

In some embodiments, a pharmaceutical composition provided herein comprises no more than 0.1% v/v, no more than 0.2% v/v, no more than 0.3% v/v, no more than 0.4% v/v, no more than 0.5% v/v, no more than 0.6% v/v, no more than 0.7% v/v, no more than 0.8% v/v, no more than 0.9% v/v, no more than 1% v/v, no more than 1.1% v/v, no more than 1.2% v/v, no more than 1.3% v/v, no more than 1.4% v/v or no more than 1.5% v/v DMSO.

In some embodiments, a composition comprising a therapeutic population of Tregs provided herein comprises no contaminants. In some embodiments, a composition comprising a therapeutic population of Tregs provided herein comprises a sufficiently low level of contaminants as to be suitable for administration, e.g., therapeutic administration, to a subject, for example a human subject. Contaminants include, for example, bacteria, fungus, mycoplasma, endotoxins or residual beads from the expansion culture. In some embodiments, a composition comprising a therapeutic population of Tregs provided herein comprises less than about 5 EU/kg endotoxins. In some embodiments, a composition comprising a therapeutic population of Tregs provided herein comprises about or less than about 100 beads per 3×10⁶ cells.

In some embodiments, a composition comprising a therapeutic population of Tregs provided herein is sterile. In some embodiments, isolation or enrichment of the cells is carried out in a closed or sterile environment, for example, to minimize error, user handling and/or contamination. In some embodiments, sterility may be readily accomplished, e.g., by filtration through sterile filtration membranes. 7.4 Methods of Treatment

Provided herein are methods of treatment comprising administering an effective amount of an expanded population of Tregs as described herein to a subject in need thereof. For example, provided herein are methods of treatment comprising administering an effective amount of an ex vivo-expanded population of Tregs as described herein, e.g., as produced by the methods presented herein, to a subject in need thereof.

Provided herein are methods of treatment comprising administering an effective amount of an expanded population of Tregs as described herein, wherein the population has been cryopreserved, to a subject in need thereof. For example, provided herein are methods of treatment comprising administering an effective amount of an ex vivo-expanded population of Tregs as described herein, e.g., as produced by the methods presented herein, wherein the population has been cryopreserved, to a subject in need thereof.

Provided herein are methods of treatment comprising administering an effective amount of an expanded population of Tregs as described herein, wherein the population has been cryopreserved, to a subject in need thereof, wherein the cryopreserved population is thawed and administered to the subject without further expansion. For example, provided herein are methods of treatment comprising administering an effective amount of an ex vivo-expanded population of Tregs as described herein, e.g., as produced by the methods presented herein, wherein the population has been cryopreserved, to a subject in need thereof, wherein the cryopreserved population is thawed and administered to the subject without further expansion.

Provided herein are methods of treatment comprising administering an effective amount of a cryopreserved composition comprising a therapeutic population of Tregs to a subject in need thereof. In some embodiments provided herein are methods for treating neurodegenerative disorders in a subject in need thereof comprising administering an effective amount of a cryopreserved composition comprising a therapeutic population of Tregs to a subject in need thereof. In certain embodiments, the cryopreserved population is thawed and administered to the subject without further expansion.

Provided herein are methods of treatment comprising administering an effective amount of a pharmaceutical composition described herein that comprises a therapeutic population of Tregs (e.g., a thawed and unexpanded form of a cryopreserved therapeutic population of Tregs described herein) to a subject in need thereof. In some embodiments, the Tregs in the pharmaceutical composition are autologous to the subject. In other embodiments, the Tregs in the pharmaceutical composition are allogeneic to the subject.

In some embodiments, the subject is diagnosed with or is suspected of having a disorder associated with Treg dysfunction. In some embodiments, the subject is diagnosed with or is suspected of having a disorder associated with Treg deficiency. In some embodiments, the subject is diagnosed with or is suspected of having a condition (e.g., an inflammatory condition) driven by a T cell response. In some embodiments, the subject is diagnosed with or is suspected of having a condition (e.g., an inflammatory condition) driven by a myeloid cell response. In some embodiments, the subject is diagnosed with or is suspected of having a condition whose symptoms are contributed to (e.g., brought on or worsened by) a myeloid cell response. Certain embodiments, the condition is an inflammatory, autoimmune or neurodegenerative disorder. In specific embodiments, the myeloid cell is a monocyte, macrophage or microglia. In certain embodiments, the myeloid cells comprise monocytes or macrophages in the periphery, outside the central nervous system.

In some embodiments the subject is diagnosed with or is suspected of having a neurodegenerative disease. In some embodiments, the subject is diagnosed with or is suspected of having Alzheimer's disease, Amyotrophic Lateral Sclerosis, Huntington's disease, Parkinson's disease, or frontotemporal dementia.

In some embodiments, the subject is diagnosed with or is suspected of having a disorder that would benefit from downregulation of the immune system.

In some embodiments, the subject is diagnosed with or suspected of having an autoimmune disease. The autoimmune disease may be, for example, systemic sclerosis (scleroderma), polymyositis, ulcerative colitis, inflammatory bowel disease, Crohn's disease, celiac disease, multiple sclerosis (MS), rheumatoid arthritis (RA), Type I diabetes, psoriasis, dermatomyositis, lupus, e.g., systemic lupus erythematosus, or cutaneous lupus, myasthenia gravis, autoimmune nephropathy, autoimmune hemolytic anemia, autoimmune cytopenia, autoimmune encephalitis, autoimmune hepatitis, autoimmune uveitis, alopecia, thyroiditis or pemphigus.

In some embodiments, the subject is diagnosed with or suspected of having heart failure or ischemic cardiomyopathy.

In some embodiments, the subject is diagnosed with or suspected of having graft-versus-host disease, e.g., after undergoing organ transplantation (such as a kidney transplantation or a liver transplantation), or after undergoing stem cell transplantation (such as hematopoietic stem cell transplantation including a bone marrow transplant).

In some embodiments, the subject is diagnosed with or suspected of having neuroinflammation. Neuroinflammation may be associated, for example, with stroke, acute disseminated encephalomyelitis (ADEM), acute optic neuritis, acute inflammatory demyelinating polyradiculoneuropathy, chronic inflammatory demyelinating polyradiculoneuropathy, Guillain-Barre syndrome, transverse myelitis, neuromyelitis optica (NMO), epilepsy, traumatic brain injury, spinal cord injury, encephalitis, central nervous system (CNS) vasculitis, neurosarcoidosis, autoimmune or post-infectious encephalitis, or chronic meningitis.

In some embodiments, the subject is diagnosed with or suspected of having a liver disorder. The live disorder may, for example, be fatty liver, e.g., nonalcoholic fatty liver disease (NAFLD), non-alcoholic steatohepatitus (NASH), primary biliary cholangitis, autoimmune hepatitis, liver cancer, liver inflammation, hepatitis A, hepatitis B, and hepatitis C. In certain embodiments, the liver disorder is NAFLD. In certain embodiments, the liver disorder is NASH.

In some embodiments, the subject is diagnosed with or suspected of having alcoholic hepatitis (AH) or alcoholic steatohepatitis (ASH).

In some embodiments, the subject is diagnosed with or suspected of having a metabolic disorder. The metabolic disorder can be, but is not limited to, fibrosis, metabolic syndrome, NAFLD, and NASH.

In some embodiments, the subject is in need of improving islet graft survival, and the method comprises administering to the subject an expanded population of Tregs as described herein or a pharmaceutical composition described herein in combination with the islet transplantation.

In some embodiments, the subject is diagnosed with or suspected of having cardo-inflammation, e.g., cardio-inflammation associated with atheroscleorosis, myocardial infarction, ischemic cardiomyopathy, with heart failure.

In some embodiments, the subject is diagnosed with or suspected of having chronic inflammatory demyelinating polyradiculoneuropathy (CIDP). In some embodiments, the subject is diagnosed with or suspected of having acute inflammatory demyelinating polyneuropathy (AIDP). In some embodiments, the subject is diagnosed with or suspected of having Guillain-Barre syndrome (GBS).

In some embodiments, the subject has had a stroke.

In some embodiments, the subject is diagnosed with or suspected of having cancer, e.g., a blood cancer.

In some embodiments, the subject is diagnosed with or suspected of having asthma.

In some embodiments, the subject is diagnosed with or suspected of having eczema.

In some embodiments, the subject is diagnosed with or suspected of having a disorder associated with overactivation of the immune system.

In some embodiments, the subject is diagnosed with or suspected of having Tregopathy. The Tregopathy may be caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.

In some embodiments, about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ CD4⁺CD25+ cells per kg of body weight of the subject are administered. In some embodiments, 1×10⁶ CD4⁺CD25+ cells (+/−10%) per kg of body weight of the subject are administered.

In some embodiments, about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ CD4⁺CD25+ cells are administered to a patient.

In some embodiments, about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 7×10⁷, about 7×10⁷ to about 8×10⁷, about 8×10⁷ to about 9×10⁷, about 9×10⁷ to about 1×10⁸, about 1×10⁸ to about 2×10⁸, about 2×10⁸ to about 3×10⁸, about 3×10⁸ to about 4×10⁸, about 4×10⁸ to about 5×10⁸, about 5×10⁸ to about 6×10⁸, about 6×10⁸ to about 7×10⁸, about 7×10⁸ to about 8×10⁸, about 8×10⁸ to about 9×10⁸, about 9×10⁸ to about 1×10⁹ CD4⁺CD25+ cells are administered to a patient in one infusion.

In some embodiments, a cryopreserved composition comprising a therapeutic population of Tregs is administered within about 30 minutes, about 1 h, about 2-3 h, about 3-4 h, about 4-5 h, about 5-6, about 6-7 h, about 7-8 h, about 8-9 h, or about 9-10 h of thawing the cryopreserved composition comprising a therapeutic population of Tregs. The cryopreserved composition comprising a therapeutic population of Tregs may be stored at about 2° C. to about 8° C. (e.g., at about 4°) between thawing and administration.

In some embodiments, one dose of a therapeutic population of Tregs or a composition comprising a therapeutic population of Tregs is administered to a subject. In some embodiments, a therapeutic population of Tregs or a composition comprising a therapeutic population of Tregs is administered more than once. In some embodiments, a therapeutic population of Tregs or a composition comprising a therapeutic population of Tregs is administered two or more times. In some embodiments, a therapeutic population of Tregs or a composition comprising a therapeutic population of Tregs is administered every 1-2 weeks, 2-3 weeks, 3-4 weeks, 4-5 weeks, 5-6 weeks, 6-7 weeks, 7-8 weeks, 8-9 weeks, 9-10 weeks, 10-11 weeks, 11-12 weeks, every 1-2 months, 2-3 months, 3-4 months, 4-5 months, 5-6 months, 6-7 months, 7-8 months, 8-9 months, 9-10 months, 10-11 months, 11-12 months, 13-14 months, 14-15 months, 15-16 months, 16-17 months, 17-18 months, 18-19 months, 19-20 months, 20-21 months, 21-22 months, 22-23 months, 23-24 months, every 1-2 years, 2-3 years, 3-4 years or 4-5 years.

In some embodiments, about 1×10⁶ Tregs per kg of body weight of the subject are administered in the first administration and the number of Tregs administered is increased in the second third and subsequent administration. In some embodiments, about 1×10⁶ Tregs per kg of body weight of the subject are administered in the first two administrations, and the number of Tregs administered is increased in every other administration thereafter (e.g., the 4^{th}, 6^{th}, 8^{th} and 10^{th} administration). Thus, for example, about 1×10⁶ Tregs per kg of body weight of the subject may be administered per month for the first and second month, and about 2×10⁶ Tregs per kg of body weight of the subject may be administered per month for the third and fourth month, and/or about 3×10⁶ cells per kg of body weight of the subject are administered per month for the fifth and sixth month.

In some embodiments, a method of treatment provide herein comprises administering a therapeutic population of autologous Tregs or a composition comprising a therapeutic population of autologous Tregs to the subject. In other embodiments, a method of treating a neurodegenerative disorder in a subject comprises administering a therapeutic population of allogeneic Tregs or a composition comprising a therapeutic population of allogeneic Tregs to the subject.

### 7.4.1. Additional Therapies

In some embodiments, In some embodiments, a subject treated in accordance with the method of treatment described herein further received one or more additional therapy or additional therapies.

In some embodiments, the subject is additionally administered IL-2. The dose of IL-2 may be about 0.5-1×10⁵IU/m², about 1-1.5×10⁵IU/m², about 1.5-2×10⁵IU/m², about 2-2.5×10⁵ IU/m², about 2.5-3×10⁵IU/m², about 3-3.5×10⁵IU/m², about 3.5-4×10⁵IU/m², about 4-4.5×10⁵ IU/m², about 4.5-5×10⁵IU/m², about 5-6×10⁵IU/m², about 6-7×10⁵IU/m², about 7-8×10⁵IU/m², about 8-9×10⁵IU/m², about 9-10×10⁵IU/m², about 10-15×10⁵IU/m², about 15-20×10⁵IU/m², about 20-25×10⁵IU/m², about 25-30×10⁵IU/m², about 30-35×10⁵IU/m², about 35-40×10⁵IU/m², about 40-45×10⁵IU/m², about 45-50×10⁵IU/m², about 50-60×10⁵IU/m², about 60-70×10⁵IU/m², about 70-80×10⁵IU/m², about 80-90×10⁵IU/m², or about 90-100×10⁵IU/m². In specific embodiments, the subject is administered 2×10⁵IU/m² of IL-2.

The IL-2 may be administered one, two or more times a month. In some embodiments, the IL-2 is administered three times a month.

In some embodiments, the IL-2 is administered subcutaneously.

The IL-2 may be administered at least 2 weeks, at least 3 weeks, or at least 4 weeks prior to the first Treg infusion.

In some embodiments, the subjected treated in accordance with the methods described herein receives one or more additional therapies are for the treatment of Alzheimer's. Addition therapies for the treatment of Alzheimer's may include acetylcholinesterase inhibitors (e.g., donepezil (Aricept®), galantamine (Razadyne®), or rivastigmine (Exelon®)) or NMDA receptor antagonists (e.g., Memantine (Akatinol®, Axura®, Ebixa®/Abixa®, Memox® and Namenda®). Additional therapies may also include anti-inflammatory agents (e.g., nonsteroidal anti-inflammatory drugs (NSAID) such as ibuprofen, indomethacin, and sulindac sulfide)), neuronal death associated protein kinase (DAPK) inhibitors such as derivatives of 3-amino pyridazine, Cyclooxygenases (COX-1 and −2) inhibitors, or antioxidants such as vitamins C and E.

In some embodiments, a subject treated in accordance with the methods described herein receives on or more additional therapies for the treatment of ALS. Additional therapies for the treatment of ALS may include Riluzole (Rilutek®) or Riluzole (Rilutek®)

In some embodiments, the therapeutic population of Tregs or the composition comprising a therapeutic composition of Tregs is administered to a subject by intravenous infusion.

### 7.4.2. Methods of Determining Treatment Effect

In some embodiments, a method of treatment provided herein results in an increase in the Treg suppressive function in the blood from baseline. In some embodiments, a method of treatment provided herein results in an increase in the Treg suppressive function in the blood from baseline to week 4, week 8, week 16, week 24, week 30 or week 36. In some embodiments, a method of treatment provided herein results in an increase in the Treg suppressive function in the blood from baseline to week 24. In some embodiments, a method of treatment provided herein results in an increase in the Treg numbers in the blood from baseline. In some embodiments, a method of treatment provided herein results in an increase in the Treg numbers in the blood from baseline to week 4, week 8, week 16, week 24, week 30 or week 36. In some embodiments, a method of treatment provided herein results in an increase in the Treg numbers in the blood from baseline to week 24.

The effect of a method of treatment provided herein may be assessed by monitoring clinical signs and symptoms of the disease to be treated.

In some embodiments, method of treatment provided herein results in a change in the Appel ALS score compared to baseline. The Appel ALS score measures overall progression of disability or altered function. In some embodiments, the Appel ALS score decreases in a subject treated in accordance with a method provided herein compared to baseline, indicating an improvement of symptoms. In other embodiments, the Appel ALS score remains unchanged ins a subject treated in accordance with a method provided herein compared to baseline.

In some embodiments, a method of treatment provided herein results in a change in the Amyotrophic Lateral Sclerosis Functional Rating Scale-revised (ALSFRS-R) score compared to baseline. The ALSFRS-R score assesses the progression of disability or altered function. In some embodiments, the ALSFRS-R score increases in a subject treated in accordance with a method provided herein compared to baseline, indicating an improvement of symptoms. In other embodiments, the Appel ALSFRS-R score remains unchanged in a subject treated in accordance with a method provided herein compared to baseline.

In some embodiments, a method of treatment provided herein results in a change in forced vital capacity (FVC; strength of muscles used with expiration) compared to baseline, where the highest number is the strongest measurement. In some embodiments, FVC increases in a subject treated in accordance with a method provided herein compared to baseline. In other embodiments, FVC remains unchanged in a subject treated in accordance with a method provided herein compared to baseline.

In some embodiments, a method of treatment provided herein results in a change in Maximum Inspiratory Pressure (MIP; strength of muscles used with inspiration) compared where the highest number is the strongest measurement. In some embodiments, MIP increases in a subject treated in accordance with a method provided herein compared to baseline. In other embodiments, MIP remains unchanged in a subject treated in accordance with a method provided herein compared to baseline.

In some embodiments, a method of treatment provided herein results in a change in Neuropsychiatric Inventory Questionnaire (NPI-Q) compared to baseline. The NPI-Q provides symptom Severity and Distress ratings for each symptom reported, and total Severity and Distress scores reflecting the sum of individual domain scores. In some embodiments, the NPI-Q score decreases in a subject treated in accordance with a method provided herein compared to baseline. In other embodiments, NPI-Q score remains unchanged in a subject treated in accordance with a method provided herein compared to baseline.

In some embodiments, a method of treatment provided herein results in a decrease in the frequency of GI symptoms, anaphylaxis or seizures compared to baseline.

In some embodiments, a method of treatment provided herein results in a change in a change in CSF amyloid and/or CSF tau protein (CSF-tau) compared to baseline. In some embodiments, the levels of CSF amyloid and/or CSF tau protein decreases in a subject treated in accordance with a method provided herein compared to baseline. In other embodiments, the levels of CSF amyloid and/or CSF tau protein remains unchanged in a subject treated in accordance with a method provided herein compared to baseline.

In some embodiments, a method of treatment provided herein results in a change in Clinical Dementia Rating (CDR) compared to baseline. The CDR rates memory, orientation, judgment and problem-solving, community affairs, home and hobbies, and personal care, and a global rating is then generated, ranging from 0-no impairment to 3-severe impairment. In some embodiments, the CDR decreases in a subject treated in accordance with the methods provided herein compared to baseline. In other embodiments, the CDR remains unchanged in a subject treated in accordance with a method provided herein compared to baseline.

In some embodiments, a method of treatment provided herein results in a change in Alzheimer's Disease Assessment Scale (ADAS)-cogl3 score compared to baseline. ADAS-cog tests cognitive performance and has an upper limit is 85 (poor performance) and lower limit is zero (best performance). In some embodiments, the ADAS-cog13 score decreases in a subject treated in accordance with a method provided herein compared to baseline. In other embodiments, the ADAS-cog13 score remains unchanged in a subject treated in accordance with a method provided herein.

The efficacy of a method of treatment described herein may be assessed at about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, about 76 weeks, about 80 weeks, about 84 weeks, about 88 weeks, about 92 weeks, about 96 weeks, about 100 weeks, at about 2-3 months, 3-4 months, 4-5 months, 5-6 months, 6-7 months, 7-8 months, 8-9 months, about 9-10 months, about 10-11 months, about 11-12 months, about 12-18 months, about 18-24 months, about 1-2 years, about 2-3 years, about 3-4 years, about 4-5 years, about 5-6 years, about 6-7 years, about 7-8 years, about 8-9 years, or about 9-10 years after initiation of treatment in accordance with the method described herein.

### 7.5 Kits

Provided herein are kits comprising a therapeutic composition of Tregs or a composition comprising a therapeutic population of Tregs provided herein.

In some embodiments, a kit provided herein comprises instructions for use, additional reagents (e.g., sterilized water or saline solutions for dilution of the compositions), or components, such as tubes, containers or syringes for collection of biological samples, processing of biological samples, and/or reagents for quantitating the amount of one or more surface markers in a sample (e.g., detection reagents, such as antibodies).

In some embodiments, the kits contain one or more containers containing a therapeutic population of Tregs or a composition comprising a therapeutic population of Tregs for use in the methods provided herein. The one or more containers holding the composition may be a single-use vial or a multi-use vial. In some embodiments, the article of manufacture or kit may further comprise a second container comprising a suitable diluent. In some embodiments, the kit contains instruction for use (e.g., dilution and/or administration) of a therapeutic population of Tregs or a composition comprising a therapeutic population of Tregs provided herein.

### 8. EXAMPLES

The example appearing in this section is provided to demonstrate illustrative embodiments of the invention. It should be appreciated by those of ordinary skill in the art that the techniques disclosed in the example represent techniques discovered to function well in the practice of the invention, and thus can be considered to constitute preferred modes for its practice. However, those of ordinary skill in the art should, in light of the present disclosure, appreciate that many changes can be made in the specific embodiments which are disclosed and still obtain a like or similar result without departing from the spirit and scope of the invention.

### 8.1 Example 1: Automated Treg Expansion

Following isolation and enrichment (CD25⁺ enrichment/CD8+CD19+depletion, for example, via CliniMACS® Plus or CliniMACS Prodigy®), the CD25⁺-enriched cells are incubated in a Quantum Cell Expansion System (Terumo BCT).

Within 24 hours of the initiation of the culturing of the isolated and enriched cells (preferably within 30 minutes following isolation and enrichment) (Day 0), the CD25⁺ cells are activated with anti-CD3/anti-CD28 beads at a 4:1 beads-to-cell ratio in the bioreactor. IL-2 and rapamycin are also added on Day 0 within 24 hours of the initiation of the culturing of the isolated and enriched cells (preferably within 30 minutes of isolation and enrichment).

The culture medium is replenished with IL-2 every 3-4 days, and IL-2 concentration is adjusted depending on cell number (i.e., the number of all cells in culture, including the enriched Treg cells). Specifically, the cells are cultured in a culture medium containing about 200 IU/mL IL-2 until the cell number reaches 600×10⁶, and then are cultured in a culture medium containing about 250 IU/mL IL-2. The culture medium also contains human AB serum (e.g., 1% or 0.5% human AB serum).

The flow rate of the extracapillary (EC) medium is also adjusted depending on cell number (i.e., the number of all cells in culture, including the enriched Treg cells). Specifically, the flow rate of the EC medium is maintained at 0 until the cell number reaches 500×10⁶, then is increased to about 0.2 mL/min and maintained at about 0.2 mL/min until the cell number reaches 750×10⁶, then is increased to about 0.4 mL/min and maintained at about 0.4 mL/min until the cell number reaches about 1,000×10⁶, then is increased to about 0.6 mL/min and maintained at about 0.6 mL/min until the cell number reaches about 1,500×10⁶, and then is increased to about 0.8 mL/min and maintained at about 0.8 mL/min. The EC medium contains rapamycin. The cells are expanded in the Quantum bioreactor from Day 1. Cell counts and viability are determined each day. Glucose and lactate levels in the culture media are also measured daily.

Before or on Day 11, if the cell expansion yields the dose of cells required (3 2.5×10⁹ cells), then the cells are harvested and cryopreserved following bead removal. If the cell expansion process has not reached dose by Day 11, then the cells are reactivated on Day 11 with anti-CD3/anti-CD28 beads at a 1:1 beads-to-cell ratio in the bioreactor. The expansion process may continue in the bioreactor from Day 12 to Day 15, as necessary. Cell counts and viability are measured each day. Once the cell expansion process yields the dose of cells needed (occurring any day between Days 12 and 15), then the cells are immediately harvested and cryopreserved following bead removal. See FIG. 1 for a corresponding process flow diagram.

### 7.2 Example 2

### 7.2.1 Treg Expansion

A series of bioreactor production runs (BioRl-BioR6) from healthy subjdct Treg expansions were performed according to the protocols described herein (see, e.g., Example 1) in media containing 1% human AB serum, and utilizing the particular parameters set out in Table 1, below. For example, as noted in Table 1, Tregs from the healthy subjects were isolated using the CliniMACS® Plus system (Miltenyi Biotec, Inc) in five runs, and were isolated using the CliniMACS® Prodigy system (Miltenyi Biotec, Inc) in one run. Tregs were expanded in a Terumo Quantum bioreactor.

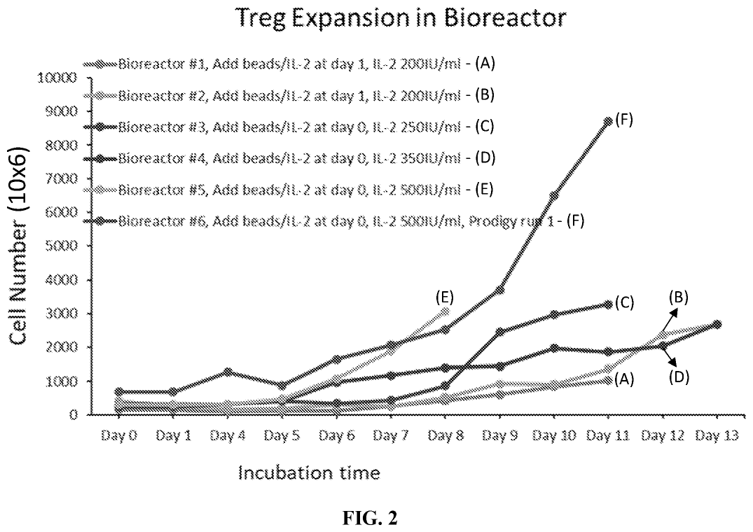

The target dose was 2.5×10⁹ total cells during these runs, which was achieved as shown in FIG. 2. Moreover, as summarized below, the resulting ex vivo expanded Tregs exhibited excellent viability and potency, even post-cryopreservation.

In FIG. 2, the final data point listed for each run represents the day the Tregs were collected. With respect to Bioreactor #6 (BioR6), for example, the starting number of cells was higher and the growth rate was faster. In addition, because the target dose before achieved over a weekend, harvesting was performed well after the target dose was attained. It is noted that only half of the Tregs isolated from the starting material were used in Bioreactor #1 (BioR1) as the other half was cultured in flasks. The rate of expansion in Bioreactor #1 was superior to that in the flasks during this single run.

TABLE 1

| | | IL-2 con. during | Day to add |
|---|---|---|---|
| | | Expansion | activation beads |
| Bioreactor Run | Isolation | (IU/ml) | and IL-2 |
| BioR1 | CliniMACS Plus | 200 | Day 1 |
| BioR2 | CliniMACS Plus | 200 | Day 1 |
| BioR3 | CliniMACS Plus | 250 | Day 0 |
| BioR4 | CliniMACS Plus | 350 | Day 0 |
| BioR5 | CliniMACS Plus | 500 | Day 0 |
| BioR6 | Prodigy | 500 | Day 0 |

7.2.2 Viability of cryopreserved Tregs from the Bioreactor after Thawing

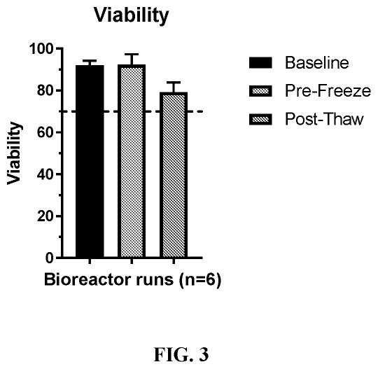

Tregs from the six completed expansions described above were harvested, underwent bead removal and then were cryopreserved in cryovials in accordance with procedures described in Section 6.2, above. The viability of the Tregs was measured at baseline (92.1%±1.89, n=6), immediately prior to freezing (92.3%±4.5, n=6) and immediately after thawing (79.2%±4.2, n=6). All thawed samples exhibited viabilities greater than 70% (Range 72%-87%), as shown in FIG. 3 (results are shown as mean±SD).

Cell characteristics in the thawed samples were assessed as described below.

7.2.3 Percentage of CD4⁺CD25+ cells

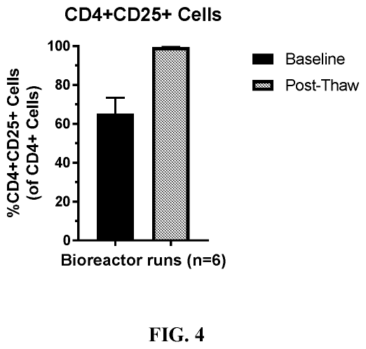

The purity of the thawed samples was determined by the percentage of CD4⁺CD25+ cells (of total CD4+ cells) via flow cytometry. The percentage of CD4⁺CD25+cells in the thawed population was 99.3%±0.4, n=6, as shown in FIG. 4 (results are shown as mean±SD.

7.2.4 Percentage of CD4⁺CD25+FoxP3+ cells

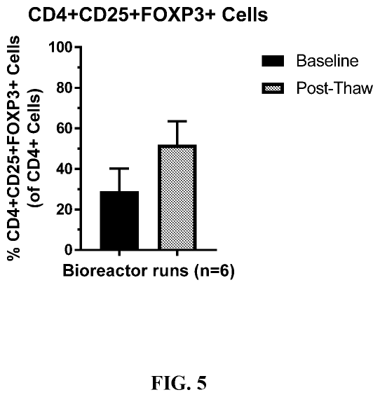

The purity of the thawed samples was determined by the percentage of CD4⁺CD25+FoxP3+ cells (of total CD4+ cells) via flow cytometry. The percentage of CD4⁺CD25+FoxP3+ cells in the thawed population was 51.8%±10.6, n=6 (Range 34 to 67%), as shown in FIG. 5 (results are shown as mean±SD).

7.2.5 Percentage of CD4⁺CD25+CD127(low)FoxP3+ cells

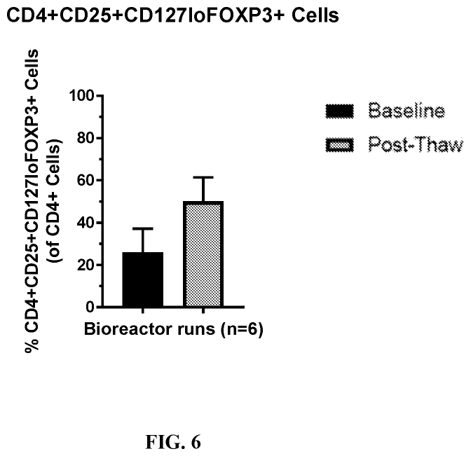

The purity of the thawed samples was determined by the percentage of CD4⁺CD25+CD127(low)FoxP3+ cells (of total CD4+ cells) via flow cytometry. The percentage of CD4⁺CD25+CD127(low)FoxP3+ cells in the thawed population was 50.1% 10.3, n=6 (Range 34 to 67%), as shown in FIG. 6 (results are reported as mean±SD).

7.2.6 Potency of cryopreserved Tregs after thawing

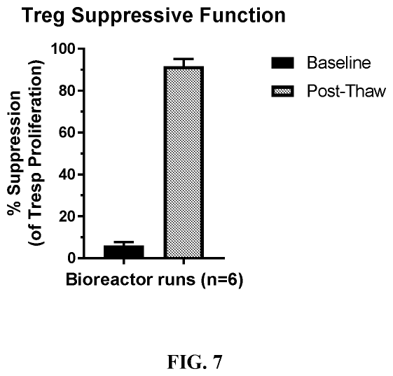

The potency of the thawed cells was determined by their suppressive capabilities on the proliferation of responder T cells. The suppressive function of the thawed population was 91.7%±3.1, n=6 (Range 88 to 97%), as shown in FIG. 7 (results are shown as mean±SD).

7.2.7 FoxP3 protein levels in cryopreserved Tregs after thawing

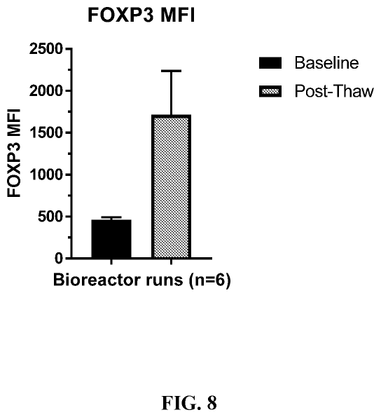

The FoxP3 protein expression on cells in the thawed samples was quantified by mean fluorescent intensity (MFI) measurements via flow cytometry. The FoxP3 MFI of the thawed population was 1714+476, n=6 (Range 1508 to 2494), as shown in FIG. 8 (results are reported as means±SD).

7.2.8 CD25 protein levels in cryopreserved Tregs after thawing

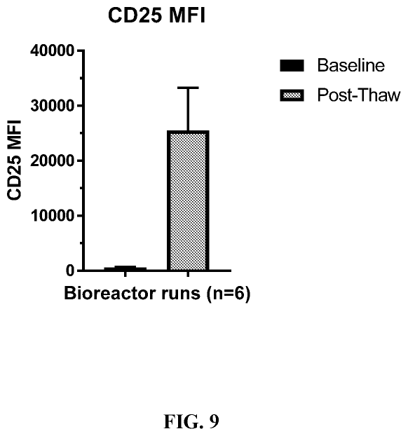

The CD25 protein expression on cells in the thawed samples was quantified by mean fluorescent intensity (MFI) measurements via flow cytometry. The CD25 MFI of the thawed population was 25471+7074, n=6 (Range 20728 to 36922), as shown in FIG. 9 (results are reported as mean±SD).

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. A method of producing a cryopreserved therapeutic population of regulatory T cells (Tregs), said method comprising the steps of:
a. enriching Tregs from a cell sample suspected of containing Tregs, to produce a baseline Treg cell population;
b. expanding the baseline Treg cell population to produce an expanded Treg cell population, wherein the baseline Treg cell population is not cryopreserved prior to the initiation of step (b), and wherein step (b) takes place in a bioreactor; and
c. cryopreserving the expanded Treg cell population to produce a cryopreserved therapeutic population of Tregs.

2. The method of claim 1, wherein step (a) comprises depleting CD8+/CD19+ cells then enriching for CD25+ cells.

3. The method of claim 1 or 2, wherein step (b) is carried out within about 30 minutes after step (a).

4. The method of any one of claims 1-3, wherein step (b) comprises culturing the Tregs in a culture medium that comprises beads coated with anti-CD3 antibodies and anti-CD28 antibodies.

5. The method of claim 4, wherein step (b) comprises adding the beads to the culture medium within about 24 hours of the initiation of the culturing.

6. The method of claim 4 or 5, wherein step (b) comprises adding the beads to the culture medium within about 30 minutes after the completion of step (a).

7. The method of any one of claims 4-6, wherein step (b) comprises adding beads coated with anti-CD3 antibodies and anti-CD28 antibodies to the culture medium about 11 days after beads coated with anti-CD3 antibodies and anti-CD28 antibodies are first added to the culture medium.

8. The method of claim 7, wherein step (b) comprises adding beads coated with anti-CD3 antibodies and anti-CD28 antibodies to the culture medium about 11 days after beads coated with anti-CD3 antibodies and anti-CD28 antibodies are first added to the culture medium, if the cell number by then has not reached a target cell number.

9. The method of claim 8, wherein the target cell number is 2.5×10⁹ cells.

10. The method of any one of claims 1-9, wherein step (b) comprises culturing the Tregs in a culture medium that comprises Interleukin-2 (IL-2).

11. The method of claim 10, wherein step (b) comprises adding IL-2 to the culture medium within about 24 hours of the initiation of the culturing.

12. The method of claim 10 or 11, wherein step (b) comprises adding IL-2 to the culture medium within about 30 minutes after the completion of step (a).

13. The method of any one of claims 10-12, wherein step (b) comprises replenishing the culture medium with IL-2 about every 3-4 days after IL-2 is first added to the culture medium.

14. The method of any one of claims 10-13, wherein step (b) comprises adjusting IL-2 concentration depending on cell number.

15. The method of claim 14, wherein step (b) comprises culturing the Tregs in a culture medium containing about 200 IU/mL IL-2 until the cell number reaches 600×10⁶, and then culturing the Tregs in a culture medium containing about 250 IU/mL IL-2.

16. The method of any one of claims 1-15, wherein step (b) comprises culturing the Tregs in a culture medium that comprises rapamycin.

17. The method of claim 16, wherein step (b) comprises adding rapamycin to the culture medium within about 24 hours of the initiation of the culturing.

18. The method of claim 16 or 17, where step (b) comprises adding rapamycin to the culture medium within about 30 minutes after the completion of step (a).

19. The method of any one of claims 1-18, wherein step (b) comprises adjusting flow rate of an extracapillary (EC) medium of the bioreactor depending on cell number.

20. The method of claim 19, wherein the extracapillary medium comprises rapamycin.

21. The method of claim 19 or 20, wherein step (b) comprises maintaining the flow rate of the EC medium at 0 until the cell number reaches 500×10⁶, then increasing the flow rate of the EC medium to about 0.2 mL/min and maintaining the flow rate of the EC medium at about 0.2 mL/min until the cell number reaches 750×10⁶, then increasing the flow rate of the EC medium to about 0.4 mL/min and maintaining the flow rate of the EC medium at about 0.4 mL/min until the cell number reaches about 1,000×10⁶, then increasing the flow rate of the EC medium to about 0.6 mL/min and maintaining the flow rate of the EC medium at about 0.6 mL/min until the cell number reaches about 1,500×10⁶, and then increasing the flow rate of the EC medium to about 0.8 mL/min and maintaining the flow rate of the EC medium at about 0.8 mL/min.

22. The method of any one of claims 1-21, wherein the cell sample is a leukapheresis cell sample.

23. The method of any one of claims 1-22, wherein step (b) is automated.

24. The method of any one of claims 1-23, wherein step (b) takes place in a closed system.

25. The method of any one of claims 1-24, wherein step (a) is automated.

26. The method of any one of claims 1-25, wherein step (a) takes place in a closed system.

27. The method of any one of claims 1-26, wherein step (a) and step (b) take place in different systems.

28. The method of claim 27, wherein the baseline Treg cell population produced by step (a) are transferred to the bioreactor in step (b) in a closed step.

29. The method of claim 27, wherein step (a) takes place in a closed system, step (b) takes place in a closed system, and the baseline Treg cell population produced by step (a) are transferred to the bioreactor in step (b) in a closed step.

30. The method of any one of claims 1-26, wherein step (a) and step (b) take place in the same system.

31. The method of claim 30, wherein the same system is a closed system.

32. The method of any one of claims 1-31, wherein the method further comprises thawing the cryopreserved therapeutic population of Tregs and, without further expansion, placing the population into a composition comprising a pharmaceutically acceptable carrier, to produce a pharmaceutical composition.

33. The method of claim 32, wherein the method further comprises administering the pharmaceutical composition to a subject.

34. The method of claim 33, wherein the Tregs in the pharmaceutical composition are autologous to the subject.

35. The method of claim 33, wherein the Tregs in the pharmaceutical composition are allogeneic to the subject.

36. The method of any one of claims 33-35, wherein the subject is a human subject.

37. A cryopreserved therapeutic population of Tregs produced by the method of any one of claims 1-31.

38. A pharmaceutical composition comprising a thawed and unexpanded form of the cryopreserved therapeutic population of Tregs of claim 37, and a pharmaceutically acceptable carrier.

39. A pharmaceutical composition produced by the method of any one of claims 32-36.

40. A method of treating a disorder associated with Treg dysfunction in a subject in need thereof, the method comprising administering to the subject the pharmaceutical composition of claim 38 or 39.

41. A method of treating a disorder associated with Treg deficiency in a subject in need thereof, the method comprising administering to the subject the pharmaceutical composition of claim 38 or 39.

42. A method of treating a disorder associated with overactivation of the immune system in a subject in need thereof, the method comprising administering to the subject the pharmaceutical composition of claim 38 or 39.

43. A method of treating an inflammatory condition driven by a T cell response in a subject in need thereof, the method comprising administering to the subject the pharmaceutical composition of claim 38 or 39.

44. A method of treating an inflammatory condition driven by a myeloid cell response in a subject in need thereof, the method comprising administering to the subject the pharmaceutical composition of claim 38 or 39.

45. The method of claim 44, wherein the myeloid cell is a monocyte, macrophage or microglia.

46. A method of treating a neurodegenerative disorder in a subject in need thereof, the method comprising administering to the subject the pharmaceutical composition of claim 38 or 39.

47. The method of claim 46, wherein the neurodegenerative disease is Amyotrophic Lateral Sclerosis (ALS), Alzheimer's disease, Parkinson's disease, frontotemporal dementia or Huntington's disease.

48. A method of treating an autoimmune disorder in a subject in need thereof, the method comprising administering to the subject the pharmaceutical composition of claim 38 or 39.

49. The method of claim 48, wherein the autoimmune disorder is polymyositis, ulcerative colitis, inflammatory bowel disease, Crohn's disease, celiac disease, systemic sclerosis (scleroderma), multiple sclerosis (MS), rheumatoid arthritis (RA), Type I diabetes, psoriasis, dermatomyosititis, systemic lupus erythematosus, cutaneous lupus, myasthenia gravis, autoimmune nephropathy, autoimmune hemolytic anemia, autoimmune cytopenia, autoimmune encephalitis, autoimmune hepatitis, autoimmune uveitis, alopecia, thyroiditis or pemphigus.

50. A method of treating graft-versus-host disease in a subject in need thereof, the method comprising administering to the subject the pharmaceutical composition of claim 38 or 39.

51. The method of claim 50, wherein the subject has received a bone marrow transplant, kidney transplant or liver transplant.

52. A method of improving islet graft survival in a subject in need thereof, comprising administering to the subject the pharmaceutical composition of claim 38 or 39 in combination with the islet transplantation.

53. A method of treating cardio-inflammation in a subject in need thereof, the method comprising administering to the subject the pharmaceutical composition of claim 38 or 39.

54. The method of claim 53, wherein the cardio-inflammation is associated with atherosclerosis, myocardial infarction, ischemic cardiomyopathy or heart failure.

55. A method of treating neuroinflammation in a subject in need thereof, the method comprising administering to the subject the pharmaceutical composition of claim 38 or 39.

56. The method of claim 55, wherein the neuroinflammation is associated with stroke, acute disseminated encephalomyelitis, acute optic neuritis, acute inflammatory demyelinating polyradiculoneuropathy, chronic inflammatory demyelinating polyradiculoneuropathy, Guillain-Barre syndrome, transverse myelitis, neuromyelitis optica, epilepsy, traumatic brain injury, spinal cord injury, encephalitis, central nervous system vasculitis, neurosarcoidosis, autoimmune or post-infectious encephalitis or chronic meningitis.

57. A method of treating a Tregopathy in a subject in need thereof, comprising administering to the subject the pharmaceutical composition of claim 38 or 39.

58. The method of claim 57, wherein the Tregopathy is caused by a FOXP3, CD25, cytotoxic T lymphocyte-associated antigen 4 (CTLA4), LPS-responsive and beige-like anchor protein (LRBA), or BTB domain and CNC homolog 2 (BACH2) gene loss-of-function mutation, or a signal transducer and activator of transcription 3 (STAT3) gain-of-function mutation.
